(19) United States
(12) Patent Application Publication
(10) Pub. No.: US 2004/0009484 A1
(43) Pub. Date: Jan. 15, 2004

(54) Methods for evaluating oligonucleotide probes of variable length

(76) Inventors: Paul K. Wolber, Los Altos, CA (US); Matthew C. Yoshikawa, Milpitas, CA (US); Chao Chen, Albany, CA (US); Stephanie B. Fulmer-Smentek, Sunnyvale, CA (US); Karen W. Shannon, Los Gatos, CA (US); Francisco J. Cifuentes, Daly City, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/195,053

(22) Filed: Jul. 11, 2002

(51) Int. Cl. C12Q 1/68; G06F 19/00; G01N 33/48; G01N 33/50

(52) U.S. Cl. 435/6; 702/20

(57) ABSTRACT

Methods are disclosed for predicting the potential of a hybridization oligonucleotide of length greater than about 20 nucleotides to hybridize to a target nucleotide sequence. The method involves evaluating predictor oligonucleotides based on one or more parameters. A subset of oligonucleotides within the predetermined number of predictor oligonucleotides is selected based on an examination of the parameter and application of a rule that rejects some of the oligonucleotides identified above. Oligonucleotides are identified in the selected subset, viewed according to order of position along the nucleotide sequence, that are in clusters along a region of the nucleotide sequence. The clusters are ranked in order of number of oligonucleotides. A hybridization oligonucleotide is selected for each cluster, in descending order of cluster rank. The selected hybridization oligonucleotide has as its central nucleotide the central nucleotide of a region of the nucleotide sequence that corresponds to the cluster. The remaining nucleotides of the hybridization oligonucleotide are added, in correspondence to the nucleotides in the nucleotide sequence that extend in one or both directions from the central nucleotide, until a hybridization oligonucleotide of predetermined length is obtained. Cross-hybridization probes are obtained based on the hybridization oligonucleotides by deletion of at least one nucleotide from the hybridization oligonucleotide wherein the deletion(s) are evenly spaced with respect to the nucleotides of the hybridization oligonucleotide and are based on the number of nucleotides in the hybridization oligonucleotide.

### BACKGROUND OF THE INVENTION

Molecular methods using DNA probes, nucleic acid hybridizations and in vitro amplification techniques are promising methods offering advantages to conventional methods used for patient diagnoses, biomedical research or basic biology research. Recent advances in such methods often include the introduction of parallelism, i.e., performing many experiments with the same effort previously used to perform a single experiment. However, the introduction of parallelism often forces changes in the methods used to design such experiments.

Nucleic acid hybridization has been employed for investigating the identity and establishing the presence of nucleic acids. Hybridization is based on complementary base pairing. When complementary single stranded nucleic acids are incubated together, the complementary base sequences pair to form double stranded hybrid molecules. The ability of single stranded deoxyribonucleic acid (ssDNA) or ribonucleic acid (RNA) to form a hydrogen bonded structure with a complementary nucleic acid sequence has been employed as an analytical tool in molecular biology research. The availability of radioactively, chemically and fluorescently labeled nucleoside triphosphates of high specific activity have made it possible to identify, isolate, and characterize various nucleic acid sequences of biological interest. Nucleic acid hybridization has great potential in diagnosing or characterizing diseased or altered tissue function associated with unique nucleic acid sequences or gene expression states. Unique nucleic acid sequences may result from genetic or environmental change in DNA by insertions, deletions, point mutations, or by acquiring foreign DNA or RNA by means of infection by bacteria, molds, fungi, and viruses. Altered gene expression states may arise from neoplastic transformation, viral infection, environmental insult or drug treatment. It is desirable to perform such experiments in parallel; earlier methods for introducing modest parallelism include Southern blots, Northern blots and slot blots.

Such blot techniques are examples of methods for detecting nucleic acids that employ nucleic acid probes that have sequences complementary to sequences in the target nucleic acid. A nucleic acid probe may be, or may be capable of being, labeled with a reporter group or may be, or may be capable of becoming, bound to a support. Detection of signal depends upon the nature of the label or reporter group. Usually, the probe is comprised of natural nucleotides such as ribonucleotides and deoxyribonucleotides and their derivatives although unnatural nucleotide mimetics such as peptide nucleic acids and oligomeric nucleoside phosphonates are also used. Commonly, binding of the probes to the target is detected by means of a label incorporated into the probe. Alternatively, the probe may be unlabeled and the target nucleic acid labeled. Binding can be detected by separating the bound probe or target from the free probe or target and detecting the label. In one approach, a sandwich is formed comprised of one probe, which may be labeled, the target and a probe that is or can become bound to a surface. Alternatively, binding can be detected by a change in the signal-producing properties of the label upon binding, such as a change in the emission efficiency of a fluorescent or chemiluminescent label. This permits detection to be carried out without a separation step. Finally, binding can be detected by labeling the target, allowing the target to hybridize to a surface-bound probe, washing away the unbound target and detecting the labeled target that remains.

Direct detection of labeled target hybridized to surface-bound probes is particularly advantageous if the surface contains a mosaic of different probes that are individually localized to discrete, known areas of the surface. Such ordered arrays containing a large number of oligonucleotide probes have been developed as tools for high throughput analyses of genotype and gene expression. Oligonucleotides synthesized on a solid support recognize uniquely complementary nucleic acids by hybridization, and arrays can be designed to define specific target sequences, analyze gene expression patterns or identify specific allelic variations. One difficulty in the design of oligonucleotide arrays is that oligonucleotides targeted to different regions of the same gene can show large differences in hybridization efficiency, presumably due to the interplay between the secondary structures of the oligonucleotides and their targets and the stability of the final probe/target hybridization product.

A method for predicting which oligonucleotides will show detectable hybridization would substantially decrease the number of iterations required for optimal array design and would be particularly useful when the total number of oligonucleotide probes on the array is limited. A method to predict oligonucleotide hybridization efficiency would also streamline the empirical approaches currently used to select potential antisense therapeutics, which are designed to modulate gene expression in vivo by hybridizing to specific messenger RNA (mRNA) molecules and inhibiting their translation into proteins.

While it is well known that the structure of the target nucleic acid affects the affinity of oligonucleotide hybridization, current methods for predicting target structures from the primary sequence fail to predict target regions accessible for oligonucleotide binding. As a result, selection of oligonucleotides for antisense reagents or oligonucleotide probe arrays has been largely empirical. As most of the target sequence is sequestered by intramolecular base pairing and not accessible for oligonucleotide binding, the process of identifying good oligonucleotides has required large numbers of low efficiency experiments.

The design and implementation of algorithms that effectively predict the ability of oligonucleotides to rapidly and avidly bind to complementary nucleotide sequences has been an important problem in molecular biology since the invention of facile methods for chemical DNA synthesis. The subsequent inventions of the polymerase chain reaction (PCR), anti-sense inhibition of gene expression and oligonucleotide array methods for performing massively parallel hybridization experiments have made the need for effective predictive algorithms even more critical.

Some previous attempts to solve the nucleic acid probe design problem include PCR primer design software applications (e.g., OLIGO®), neural networks, PCR primer design applications that search for sequences that possess minimal ability to cross-hybridize with other targets present in a sample (e.g., HYBsimulator™), and approaches that attempt to predict the efficiency of antisense sequence suppression of m-RNA translation from a combination of predicted nucleic acid duplex melting temperature and predicted target strand structure. The methods that predict effective oligonucleotide primers for performing PCR from DNA templates work well for that application where relatively stringent conditions are employed. This is because PCR experimental design greatly simplifies the prediction problem: hybridization is performed at high temperature, at relatively low ionic strength and in the presence of a large molar excess of oligonucleotide. Under these conditions, the oligonucleotide and target secondary structures are relatively unimportant.

Unfortunately, none of these conditions applies to oligonucleotide arrays, which are usually hybridized under relatively non-denaturing conditions (to boost signal), using a relatively low concentration of probe molecules linked to a surface, or to anti-sense suppression of gene expression, which takes place in vivo. Oligonucleotide arrays can contain hundreds of thousands of different sequences and conditions are chosen to allow the oligonucleotide with the lowest melting temperature to hybridize efficiently. These “lowest common denominator” conditions are usually relatively non-denaturing and secondary structure constraints become significant. Accordingly, the above applications require new predictive methods that are capable of estimating the effects of oligonucleotide and target structure on hybridization efficiency. For these reasons, current algorithms for designing PCR primer oligonucleotides fail badly when applied to the problems of oligonucleotide array or anti-sense oligonucleotide design.

Until recently, the most effective approach for identifying oligonucleotides with good hybridization efficiency has been an empirical one. Such an approach involves the synthesis of every possible oligonucleotide probe for a given target nucleotide sequence. Arrays are formed that include the above oligonucleotide probes. Hybridization experiments are carried out to determine which of the oligonucleotide probes exhibit good hybridization efficiencies. Examples of such an approach are found in Lockhart, et al., Nature Biotech., infra, and L. Wodicka, et al., Nature Biotechnology, infra. One major drawback to this approach is the vast number of oligonucleotides that must be synthesized in order to achieve a satisfactory result. Typically, about 2%-5% of the test probes synthesized yield acceptable signal levels.

The use of neural networks for oligonucleotide design has also been investigated. Neural networks are easily taught with real data; they therefore afford a general approach to many problems. However, their performance is limited by the “senses” that they are given. An analogy works best here: the human brain is an astoundingly capable neural network, but a blind person cannot be taught to reliably distinguish colors by smell. In addition, a large amount of data is required to adequately teach a neural network to perform its job well. A comprehensive database for either oligonucleotide array design or anti-sense suppression of gene expression has not been made available. For these reasons, the performance reported to-date of neural network solutions against the probe design problem is mediocre.

Finally, approaches that have attempted to use target nucleic acid folding calculations to predict experimental results inferred to depend upon hybridization efficiency (e.g. anti-sense suppression of m-RNA translation) have so far only demonstrated that the predictions of current nucleic acid folding calculations correlate poorly with observed behavior. The probable reason for this is that the structures predicted by such programs for long sequences are poor predictors of chemical reality; the results of experiments that attempt to confirm the predictions of such calculations support this assessment.

Recently, a method or algorithm was described for predicting oligonucleotides specific for a target nucleic acid where the oligonucleotides exhibit a high potential for hybridization (Shannon, et al., Method for evaluating oligonucleotide probe sequences, U.S. Pat. No. 6,251,588 (2001)). The algorithm uses parameters of the oligonucleotide and the oligonucleotide:target nucleotide sequence duplex, which can be readily predicted from the primary sequences of the target polynucleotide and candidate oligonucleotides. In the method, oligonucleotides are filtered based on one or more of these parameters, then further filtered based on the sizes of clusters of oligonucleotides. The basic steps involved in the disclosed method involve parsing a sequence that is complementary to a target nucleotide sequence into a set of overlapping oligonucleotide sequences, calculating one or more parameters for each of the oligonucleotide sequences with respect to its hybridization to the target nucleotide sequence, filtering the oligonucleotide sequences based on the values for each parameter, filtering the oligonucleotide sequences based on the length of contiguous sequence elements and ranking the contiguous sequence elements based on their length. Certain oligonucleotides within the longest contiguous sequence elements generally showed the highest hybridization efficiencies.

In many assays there may be one or more non-target nucleic acids present that have a nucleotide sequence closely related to that of the target sequence differing by only a few, e.g., one to five nucleotides. In such cases the non-target polynucleotide may then interfere with the assay by hybridizing with at least some of the target probe to produce false qualitative or quantitative results. This problem is particularly acute where the probe sequence is selected to permit assaying of various genes within a multigene family, each member of which contains a sequence closely related to the target nucleotide sequence. In analysis by array technology there is the concern that cross-hybridization may occur, which would result in false positive signals.

Approaches have been suggested for alleviating some of the above concerns. One technique involves placing on an array intentionally mismatched control probes as well as the actual probe of interest. A mismatched probe has one or more base substitutions. By observing the signal for the original probe versus the mismatched probes one can gauge specificity and perhaps even correct for cross-hybridization by subtracting some fraction of the mismatch probe signal from the signal generated by the probe of interest. In a particular approach probes are generated by constructing all possible one base substitutions at a specific position near the center of the probe and synthesizing them next to the probe of interest. However, this mismatch strategy is relatively arbitrary and multiplies by 5 the number of array locations required to evaluate the performance of a single probe. In some arrays, the percentage of array locations devoted to mismatch probes is decreased by choosing a single base substitution. However, this choice is even more arbitrary than synthesizing all possibilities at a single position.

Recently, methods, reagents and kits were disclosed for selecting target-specific oligonucleotide probes, which may be used in analyzing a target nucleic acid sequence (see, for example, U.S. patent application Ser. No. 09/350,969 filed Jul. 9, 1999, and Agilent Technologies Inc. (Palo Alto, Calif.) brochure dated Nov. 1, 2001, entitled “Development of an in situ synthesized oligonucleotide microarray for gene expression monitoring of the budding yeast Saccharomyces cerevisiae,” by Stephanie Fulmer-Smentek, et al.). In the method a cross-hybridization oligonucleotide probe is identified based on a candidate target-specific oligonucleotide probe for the target nucleic acid sequence. The cross-hybridization oligonucleotide probe measures the extent of occurrence of a cross-hybridization event having a predetermined probability. Cross-hybridization results are determined employing the cross-hybridization oligonucleotide probe and the target-specific oligonucleotide probe. The target-specific oligonucleotide probe is selected or rejected for the set based on the cross-hybridization results. The process for identifying and selecting the minimum number of cross-hybridization oligonucleotide probes may be carried out using different approaches such as mismatch probe design by homology, mismatch probes that incorporate base combinations, mismatch probes that delete bases, mismatch probes that insert bases, and combinations thereof.

### SUMMARY OF THE INVENTION

One embodiment of the present invention is a method for predicting the potential of a hybridization oligonucleotide of length greater than about 20 nucleotides to hybridize to a target nucleotide sequence. A predetermined number of unique oligonucleotides of at least about 20 nucleotides in length within a nucleotide sequence of at least about 30 nucleotides in length that is hybridizable with the target nucleotide sequence is identified. The oligonucleotides are chosen to sample a length of the nucleotide sequence. At least one parameter is determined and evaluated for the oligonucleotides. The parameter is predictive of the ability of the oligonucleotides to hybridize to the target nucleotide sequence. A subset of oligonucleotides is selected within the predetermined number of unique oligonucleotides based on an examination of the parameter and application of a rule that rejects some of the oligonucleotides of step (b). Oligonucleotides are identified in the selected subset, viewed according to order of position along the nucleotide sequence, that are in clusters along a region of the nucleotide sequence and that identify a contig in the nucleotide sequence. A hybridization oligonucleotide is selected for each cluster where the hybridization oligonucleotide comprises at least a portion of the nucleotide sequence of the contig wherein the hybridization oligonucleotide is different from any of the oligonucleotides in (d) which identify the contig.

Another embodiment of the present invention is a method for predicting the potential of a hybridization oligonucleotide of length greater than about 20 nucleotides to hybridize to a target nucleotide sequence. A predetermined number of predictor oligonucleotides of at least about 20 nucleotides in length is identified within a nucleotide sequence of at least about 30 nucleotides in length that is hybridizable with the target nucleotide sequence. The predictor oligonucleotides are chosen to sample the entire length of the nucleotide sequence. At least one parameter that is independently predictive of the ability of each of the predictor oligonucleotides to hybridize to the target nucleotide sequence is determined and evaluated for each of the predictor oligonucleotides. A subset of oligonucleotides within the predetermined number of predictor oligonucleotides is selected based on an examination of the parameter and application of a rule that rejects some of the oligonucleotides identified above. Oligonucleotides are identified in the selected subset, viewed according to order of position along the nucleotide sequence, that are in clusters along a region of the nucleotide sequence. The clusters are ranked in order of number of oligonucleotides in each cluster from greatest number to least number of oligonucleotides. A hybridization oligonucleotide is selected for each cluster, in descending order of cluster rank. The selected hybridization oligonucleotide has as its central nucleotide the central nucleotide of a region of the nucleotide sequence that corresponds to the cluster. The remaining nucleotides of the hybridization oligonucleotide are added, in correspondence to the nucleotides in the nucleotide sequence that extend in one or both directions from the central nucleotide, until a hybridization oligonucleotide of predetermined length is obtained and a predetermined number of hybridization oligonucleotides are obtained, one from each cluster. The predetermined length of the hybridization oligonucleotide is greater than the length of any of the oligonucleotides in the cluster. The higher the rank of the cluster, the higher is the hybridization potential of the hybridization oligonucleotide for the target nucleotide sequence. The hybridization oligonucleotide is different from any of the oligonucleotides in the cluster.

Another embodiment of the present invention is a method for predicting the potential of a hybridization oligonucleotide of length greater than about 20 nucleotides to hybridize to a target nucleotide sequence. A set of overlapping predictor oligonucleotides of at least about 20 nucleotides in length is identified within a nucleotide sequence of at least about 30 nucleotides in length that is complementary to the target nucleotide sequence. For each of the predictor oligonucleotides at least two parameters that are independently predictive of the ability of each of the oligonucleotides to hybridize to the target nucleotide sequence are determined and evaluated. The parameters are poorly correlated with respect to one another. A subset of oligonucleotides within the predetermined number of predictor oligonucleotides is selected based on an examination of the parameter and application of a rule that rejects some of the oligonucleotides of identified above. Oligonucleotides in the selected subset, viewed according to order of position along the nucleotide sequence, that are in clusters along a region of the nucleotide sequence are identified. The clusters are ranked in order of number of oligonucleotides in each cluster from greatest number to least number of oligonucleotides. A hybridization oligonucleotide that has as its central nucleotide the central nucleotide of a region of the nucleotide sequence that corresponds to the cluster is selected for each cluster, in descending order of cluster rank. The remaining nucleotides of the hybridization oligonucleotide are added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster. The predetermined length of the hybridization oligonucleotide is greater than the length of any of the oligonucleotides in the cluster. The higher the rank of the cluster, the higher is the hybridization potential of the hybridization oligonucleotide for the target nucleotide sequence.

Another embodiment of the present invention is a method for predicting the potential of a hybridization oligonucleotide of at least about 20 nucleotides in length to hybridize to a complementary target nucleotide sequence. A set of overlapping predictor oligonucleotides of at least about 20 nucleotides in length and of identical length N and spaced one nucleotide apart is obtained from a nucleotide sequence at least about 30 nucleotides in length and complementary to the target nucleotide sequence. The set comprises L−N+1 predictor oligonucleotides. For each of the predictor oligonucleotides the following parameters are determined and evaluated: (i) the predicted melt temperature of the duplex of the oligonucleotide and the target nucleotide sequence corrected for salt concentration and (ii) predicted free energy of the most stable intramolecular structure of the oligonucleotide at the temperature of hybridization of each of the oligonucleotides with the target nucleotide sequence. A subset of oligonucleotides within the predetermined number of predictor oligonucleotides is selected based on an examination of the parameter and application of a rule that rejects some of the oligonucleotides of identified above. Oligonucleotides are identified in the selected subset, viewed according to order of position along the nucleotide sequence, that are in clusters along a region of the nucleotide sequence. The clusters are ranked in order of number of oligonucleotides in each cluster from greatest number to least number of oligonucleotides. A selection of a hybridization oligonucleotide is made for each cluster in descending order of cluster rank. The hybridization oligonucleotide has as its central nucleotide the central nucleotide of a region of the nucleotide sequence that corresponds to the cluster. The remaining nucleotides of the hybridization oligonucleotide are added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster. The predetermined length of the hybridization oligonucleotide is greater than the length of any of the oligonucleotides in the cluster. The higher the rank of the cluster, the higher is the hybridization potential of the hybridization oligonucleotide for the target nucleotide sequence.

Another embodiment of the present invention is a computer-based method for predicting the potential of a hybridization oligonucleotide of length greater than about 20 nucleotides to hybridize to a target nucleotide sequence. The steps of the aforementioned methods are carried out under computer control.

Another embodiment of the present invention is a computer system for conducting a method for predicting the potential of a hybridization oligonucleotide to hybridize to a target nucleotide sequence. The system comprises (a) input means for introducing a target nucleotide sequence into the computer system, (b) means for determining a number of predictor oligonucleotide sequences that are within a nucleotide sequence that is hybridizable with the target nucleotide sequence, the predictor oligonucleotide sequences being chosen to sample the entire length of the nucleotide sequence, (c) memory means for storing the predictor oligonucleotide sequences, (d) means for controlling the computer system to carry out a determination and evaluation for each of the predictor oligonucleotide sequences a value for at least one parameter that is independently predictive of the ability of each of the predictor oligonucleotide sequences to hybridize to the target nucleotide sequence, (e) means for storing the parameter values, (f) means for controlling the computer to carry out an identification from the stored parameter values a subset of oligonucleotide sequences within the number of predictor oligonucleotide sequences based on the evaluation of the parameter, (g) means for storing the subset of oligonucleotides, (h) means for controlling the computer to carry out an identification of oligonucleotide sequences in the subset that are clustered along a region of the nucleotide sequence that is hybridizable to the target nucleotide sequence, (i) means for storing the oligonucleotide sequences in the subset, (j) means for outputting data relating to the oligonucleotide sequences in the subset, (k) means for ranking the clusters in order of number of oligonucleotides in each cluster from greatest number to least number of oligonucleotides, (l) means for selecting for each cluster, in descending order of cluster rank, a hybridization oligonucleotide that has as its central nucleotide the central nucleotide of a region of the nucleotide sequence that corresponds to the cluster, the remaining nucleotides of the hybridization oligonucleotide being added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster, wherein the higher the rank of the cluster, the higher the hybridization potential of the hybridization oligonucleotide for the target nucleotide sequence and wherein the predetermined length of the hybridization oligonucleotide is greater than the length of any of the oligonucleotides in the cluster, and (m) means for outputting data relating to the hybridization oligonucleotides.

Another embodiment of the present invention is a method for selecting a cross-hybridization oligonucleotide probe in length for use in conjunction with a hybridization oligonucleotide of at least about 20 nucleotides for analyzing a target nucleotide sequence. A hybridization oligonucleotide of at least 20 nucleotides in length that is specific for the target nucleotide sequence is identified. A cross-hybridization oligonucleotide probe is selected based on the above hybridization oligonucleotide by a process comprising deletion of at least one nucleotide from the hybridization oligonucleotide wherein the deletion(s) are evenly spaced with respect to the nucleotides of the hybridization oligonucleotide.

Another embodiment of the present invention is a method for selecting a hybridization oligonucleotide of at least about 20 nucleotides in length for hybridization to a target nucleotide sequence and for selecting a cross-hybridization probe corresponding to the hybridization oligonucleotide. A predetermined number of predictor oligonucleotides of at least about 20 nucleotides in length is identified within a nucleotide sequence of at least about 30 nucleotides in length that is hybridizable with the target nucleotide sequence. The predictor oligonucleotides are chosen to sample the entire length of the nucleotide sequence. At least one parameter that is independently predictive of the ability of each of the predictor oligonucleotides to hybridize to the target nucleotide sequence is determined and evaluated for each of the oligonucleotides above. A subset of oligonucleotides within the predetermined number of predictor oligonucleotides is selected based on an examination of the parameter and application of a rule that rejects some of the oligonucleotides identified above. Oligonucleotides are identified in the selected subset, viewed according to order of position along the nucleotide sequence, that are in clusters along a region of the nucleotide sequence. The clusters are ranked in order of number of oligonucleotides in each cluster from greatest number to least number of oligonucleotides. A selection is made for each cluster, in descending order of cluster rank, of a hybridization oligonucleotide that has as its central nucleotide the central nucleotide of a region of the nucleotide sequence that corresponds to the cluster. The remaining nucleotides of the hybridization oligonucleotide are added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster. The predetermined length of the hybridization oligonucleotide is greater than the length of any of the oligonucleotides in the cluster. The higher the rank of the cluster, the higher is the hybridization potential of the hybridization oligonucleotide for the target nucleotide sequence. A cross-hybridization oligonucleotide probe is selected based on the above hybridization oligonucleotide by a process comprising deletion of at least one nucleotide from the hybridization oligonucleotide wherein the deletion(s) are evenly spaced with respect to the nucleotides of the hybridization oligonucleotide and the number of deletions is based on the length of the cross-hybridization oligonucleotide probe.

Another embodiment of the present invention is a computer-based method for selecting a hybridization oligonucleotide of at least about 20 nucleotides in length for hybridization to a target nucleotide sequence and for selecting a cross-hybridization probe corresponding to the hybridization oligonucleotide. The aforementioned steps are carried out under computer control.

Another embodiment of the present invention is a method for selecting a set of target-specific oligonucleotide probes of at least about 20 nucleotides in length for use in analyzing a target nucleotide sequence. A cross-hybridization oligonucleotide probe is identified based on the target nucleic acid sequence by a process comprising deletion of at least one nucleotide from a single hybridization oligonucleotide. The deletion(s) are evenly spaced with respect to the nucleotides of the hybridization oligonucleotide and the number of deletions is based on the length of the cross-hybridization oligonucleotide probe. Cross-hybridization results are determined employing the cross-hybridization oligonucleotide probe and target-specific oligonucleotide probe. Target-specific oligonucleotide probes are selected for the set based on the cross-hybridization results, and target-specific probe signals may be corrected for cross hybridization by subtracting some percentage of each cross-hybridization control probe signal from its associated specific probe signal.

Another embodiment of the present invention is a computer-based method for selecting a set of target-specific oligonucleotide probes of at least about 20 nucleotides in length for use in analyzing a target nucleotide sequence. Under computer control a cross-hybridization oligonucleotide probe is identified based on the target nucleic acid sequence by a process comprising deletion of at least one nucleotide from a single hybridization oligonucleotide. The deletion(s) are evenly spaced with respect to the nucleotides of the hybridization oligonucleotide and the number of deletions is based on the length of the cross-hybridization oligonucleotide probe. Cross-hybridization results are determined under computer control employing the cross-hybridization oligonucleotide probe and target-specific oligonucleotide probe. The target-specific oligonucleotide probe is selected or rejected for the set based on the cross-hybridization results, and target-specific probe signal may be corrected for cross hybridization by subtracting some percentage of the cross-hybridization control probe signal from the specific probe signal.

Another embodiment of the present invention is a method for detecting differences between an individual sequence and a known reference sequence. A labeled individual sequence, a surface bound reference oligonucleotide probe based on the known reference sequence and a set of surface bound deletion oligonucleotide probes of at least about 20 nucleotides in length are combined under hybridization conditions. The set of deletion oligonucleotide probes is prepared by a process comprising deletion of at least one nucleotide from a single hybridization oligonucleotide. The deletion(s) are evenly spaced with respect to the nucleotides of the hybridization oligonucleotide and the number of deletions is based on the length of the cross-hybridization oligonucleotide probe. Hybridization ratios are determined for the set of deletion oligonucleotide probes with respect to the reference oligonucleotide probe. The hybridization ratios are related to the presence or absence of differences between the individual sequence and the reference sequence.

Another embodiment of the present invention is an addressable array comprising a support having a surface, a spot on the surface having bound thereto an oligonucleotide probe specific for a target nucleic acid sequence and at least one spot on the surface having bound thereto a cross-hybridization oligonucleotide probe. The cross-hybridization oligonucleotide probe measures the extent of the occurrence of a cross-hybridization event of a predetermined probability between an interfering nucleic acid sequence and the oligonucleotide probe specific for a target nucleic acid sequence. The cross-hybridization oligonucleotide probe is selected by a process comprising deletion of at least one nucleotide from a single hybridization oligonucleotide wherein the deletion(s) are evenly spaced with respect to the nucleotides of the hybridization oligonucleotide and the number of deletions is based on the length of the cross-hybridization oligonucleotide probe.

Another embodiment of the present invention is a method for predicting the potential of a hybridization oligonucleotide of at least about 20 nucleotides in length to hybridize to a complementary target nucleotide sequence. A set of overlapping oligonucleotides of at least about 20 nucleotides in length and of identical length N and spaced S nucleotides apart is obtained from a nucleotide sequence at least about 30 nucleotides in length L and complementary to the target nucleotide sequence. The set comprises 1+Int[(L−N)/S] oligonucleotides, wherein “Int” is the integer part of the indicated quotient. The hybridization of the oligonucleotides with the target nucleotide sequence is determined experimentally and evaluated for each of the oligonucleotides. A subset of oligonucleotides within the predetermined number of oligonucleotides is selected based on the evaluation and application of a rule that rejects some of the oligonucleotides of the previous step. Oligonucleotides are identified in the selected subset, viewed according to order of position along the nucleotide sequence, that are in clusters along a region of the nucleotide sequence. The clusters are ranked in order of number of oligonucleotides in each cluster from greatest number to least number of oligonucleotides. A hybridization oligonucleotide that has as its central nucleotide the central nucleotide of a region of the nucleotide sequence that corresponds to the cluster is selected for each cluster, in descending order of cluster rank. The remaining nucleotides of the hybridization oligonucleotide are added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster. The predetermined length of the hybridization oligonucleotide is greater than the length of any of the oligonucleotides in the cluster. The higher the rank of the cluster, the higher is the hybridization potential of the hybridization oligonucleotide for the target nucleotide sequence. In one approach the method comprises synthesizing the oligonucleotides and experimentally testing the hybridization of the oligonucleotides with an array comprising the target nucleotide sequence.

### BRIEF DESCRIPTION OF THE DRAWINGS

FIG. 1 depicts one embodiment of the method of the present invention by way of illustration and not limitation.

FIG. 2 depicts another embodiment of the method of the present invention by way of illustration and not limitation.

FIG. 3 depicts another embodiment of the method of the present invention by way of illustration and not limitation.

FIG. 4 depicts another embodiment of the method of the present invention by way of illustration and not limitation.

FIG. 5 depicts another embodiment of the method of the present invention by way of illustration and not limitation.

FIG. 6 is a graph depicting a comparison of the sensitivity prediction in accordance with the present invention with the results of an experiment in which pure GCN4 target was hybridized to an array bearing a tiling pattern of 60-mer probes (3 nucleotide spacing) to GCN4.

FIG. 7 is a graph depicting a comparison of the specificity prediction in accordance with the present invention with the results of an experiment in which pure yeast gcn4/gcn4 knockout strain was hybridized to an array.

FIG. 8 is a graph depicting a comparison of the accuracy of detecting the ratio of a spiked-in transcript using 25-mer probes or the 60-mer probes extended from them in accordance with the present invention.

FIG. 9 is a graph illustrating the selection of 60-mer probes from empirically tested 25-mer probes in accordance with the present invention.

### DETAILED DESCRIPTION OF THE INVENTION

The invention is directed to methods and/or algorithms for predicting hybridization oligonucleotides, i.e., oligonucleotides that are specific for a nucleic acid target, where the hybridization oligonucleotides exhibit a high potential for hybridization and where the hybridization oligonucleotides are at least about 30 nucleotides in length. The algorithm uses parameters of certain oligonucleotides, referred to herein as predictor oligonucleotides, and the duplexes formed between the predictor oligonucleotides and a target nucleotide sequence. The parameters can be readily predicted from the primary sequences of the target polynucleotide and the predictor oligonucleotides. In the methods of the present invention, predictor oligonucleotides are filtered based on one or more of these parameters. The sizes of the clusters are then ranked. In one embodiment hybridization oligonucleotides are determined by selecting for each cluster, in order of its cluster rank, an oligonucleotide of predetermined length that spans at least the entire length of the contiguous region of a nucleotide sequence corresponding to the cluster. In another embodiment an oligonucleotide of predetermined length is selected that spans at least the entire length of one-half of such contiguous region from a central nucleotide plus a sequence of nucleotides that is adjacent said contiguous region. In another embodiment an oligonucleotide of predetermined length is selected that spans at least one-half of such contiguous region plus at least a portion of the remaining half of such contiguous region plus a sequence of nucleotides that is adjacent said contiguous region.

The methods or algorithms of the present invention may be carried out using either relatively simple user-written subroutines or publicly available stand-alone software applications (e.g., dynamic programming algorithm for calculating self-structure free energies of oligonucleotides). The parameter calculations may be orchestrated and the filtering algorithms may be implemented using any of a number of commercially available computer programs as a framework such as, e.g., Microsoft® Excel spreadsheet, Microsoft® Access relational database and the like.

The basic steps involved in the present methods involve parsing a nucleotide sequence that is complementary to a target nucleotide sequence into a set of overlapping predictor oligonucleotide sequences, calculating one or more parameters for each of the predictor oligonucleotide sequences with respect to its hybridization to the target nucleotide sequence, filtering the predictor oligonucleotide sequences based on the values for each parameter, determining the number of predictor oligonucleotide sequences that are in clusters based on the length of contiguous sequence elements, ranking the clusters according to the number of predictor oligonucleotide sequences contained therein, and determining an oligonucleotide that spans at least the entire region of the contiguous sequence elements (or contigs) thereby selecting a hybridization oligonucleotide of a predetermined length of at least 30 nucleotides. The process is repeated for each cluster in descending order of rank until a set of hybridization oligonucleotides of predetermined length is selected. The length of the hybridization oligonucleotides in the set may be the same or it may be varied by including additional nucleotides as discussed herein. We have found that hybridization oligonucleotides obtained in this manner generally show the highest hybridization efficiencies.

The present invention also includes methods for designing matched specificity control probes or cross-hybridization oligonucleotide probes for each potential hybridization oligonucleotide or measurement probe. In this way, experimental evaluation of probe performance may be accomplished, based on the difference of the measurement and specificity control probe signals and the ratio of those signals. In both cases larger is better, i.e., the larger the difference and the larger the ratio, the better the performance of the hybridization probe. We have found that, for longer hybridization probes, cross-hybridization oligonucleotide probes may be identified by internal deletions from the hybridization oligonucleotide probe that are evenly spaced along the probe and at a frequency of one deletion for about every 10 to 25 nucleotides in the hybridization oligonucleotide probe.

Terminology

Before proceeding further with a description of the specific embodiments of the present invention, a number of terms will be defined.

The term “polynucleotide” or “nucleic acid” refers to a compound or composition that is a polymeric nucleotide or nucleic acid polymer. The polynucleotide may be a natural compound or a synthetic compound. The polynucleotide can have from about 2 to 5,000,000 or more nucleotides. The larger polynucleotides are generally found in the natural state. In an isolated state the polynucleotide can have about 10 to 50,000 or more nucleotides, usually about 100 to 20,000 nucleotides. It is thus obvious that isolation of a polynucleotide from the natural state often results in fragmentation. It may be useful to fragment longer target nucleic acid sequences, particularly RNA, prior to hybridization to reduce competing intramolecular structures.

The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, cDNA, and the like.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method.

The nucleic acids may be generated by in vitro replication and/or amplification methods such as the Polymerase Chain Reaction (PCR), asymmetric PCR, the Ligase Chain Reaction (LCR), transcriptional amplification by an RNA polymerase, and so forth. The nucleic acids may be either single-stranded or double-stranded. Single-stranded nucleic acids are preferred because they lack complementary strands that compete for the oligonucleotide probes during the hybridization step of the method of the invention. A nucleic acid may be treated to render it denatured or single stranded by treatments that are well known in the art and include, for instance, heat or alkali treatment, or enzymatic digestion of one strand.

The phrase “target nucleotide sequence” or “target nucleic acid sequence” or “target polynucleotide” refers to a sequence of nucleotides to be identified, detected or otherwise analyzed, usually existing within a portion or all of a polynucleotide. In the present invention the identity of the target nucleotide sequence may be known to an extent sufficient to allow preparation of various sequences hybridizable with the target nucleotide sequence and of oligonucleotides, such as probes and primers, and other molecules necessary for conducting methods in accordance with the present invention, related methods and so forth.

The target sequence usually contains from about 10 to 5,000 or more nucleotides, preferably 50 to 1,000 nucleotides. The target nucleotide sequence is generally a fraction of a larger molecule or it may be substantially the entire molecule such as a polynucleotide as described above. The minimum number of nucleotides in the target nucleotide sequence is selected to assure that the presence of a target polynucleotide in a sample is a specific indicator of the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g. transcription of a DNA template into RNA) and the efficiency of identification, detection, amplification, and/or other analysis of the target nucleotide sequence, where appropriate.

It is to be noted that the usage of the terms “probe” and “target” in the literature may vary. For example, when describing non-homogeneous diagnostic assays, the term “probe” may be used to refer to an immobilized or surface-bound species, and the term target may be used to refer to a species in solution (the “target” of the assay). Such usage of the terms is the opposite of the usage sometimes seen in the molecular biology literature. The present application uses the diagnostic assay definitions of the terms “probe” and “target” as discussed herein.

The term “oligonucleotide” refers to a polynucleotide, usually single stranded, either a synthetic polynucleotide or a naturally occurring polynucleotide. The length of an oligonucleotide is generally governed by the particular role thereof, such as, for example, probe, primer, predictor and the like. Various techniques can be employed for preparing an oligonucleotide. Such oligonucleotides can be obtained by biological synthesis or by chemical synthesis. For short oligonucleotides (up to about 100 nucleotides), chemical synthesis will frequently be more economical as compared to biological synthesis. In addition to economy, chemical synthesis provides a convenient way of incorporating low molecular weight compounds and/or modified bases during specific synthesis steps. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. Methods of oligonucleotide synthesis include phosphotriester and phosphodiester methods (Narang, ET al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al., Proc. Nat. Acad. Sci. USA (1994) 91:5022-5026.

Oligonucleotides may be employed, for example, as oligonucleotide probes or primers. The term “oligonucleotide probe” refers to an oligonucleotide employed to bind to a portion of a polynucleotide such as another oligonucleotide or a target nucleotide sequence. The design, including the length, and the preparation of the oligonucleotide probes are generally dependent upon the sequence to which they bind and their function in the methods of the invention.

The phrase “nucleoside triphosphates” refers to nucleosides having a 5′-triphosphate substituent. The nucleosides are pentose sugar derivatives of nitrogenous bases of either purine or pyrimidine derivation, covalently bonded to the 1′-carbon of the pentose sugar, which is usually a deoxyribose or a ribose. The purine bases include adenine (A), guanine (G), inosine (I), and derivatives and analogs thereof. The pyrimidine bases include cytosine (C), thymine (T), uracil (U), and derivatives and analogs thereof. Nucleoside triphosphates include deoxyribonucleoside triphosphates such as the four common deoxyribonucleoside triphosphates dATP, dCTP, dGTP and dTTP and ribonucleoside triphosphates such as the four common triphosphates rATP, rCTP, rGTP and rUTP. The term “nucleoside triphosphates” also includes derivatives and analogs thereof, which are exemplified by those derivatives that are recognized and polymerized in a similar manner to the underivatized nucleoside triphosphates.

The term “nucleotide” or “nucleotide base” or “base” refers to a base-sugar-phosphate combination that is the monomeric unit of nucleic acid polymers, i.e., DNA and RNA. The term as used herein includes modified nucleotides. In general, the term refers to any compound containing a cyclic furanoside-type sugar (β-D-ribose in RNA and β-D-2′-deoxyribose in DNA), which is phosphorylated at the 5′ position and has either a purine or pyrimidine-type base attached at the C-1′ sugar position via a β-glycosol C1′-N linkage. The nucleotide may be natural or synthetic.

The term “DNA” refers to deoxyribonucleic acid.

The term “RNA” refers to ribonucleic acid.

The term “nucleoside” refers to a base-sugar combination or a nucleotide lacking a phosphate moiety.

The terms “hybridization (hybridizing)” and “binding” in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like.

The term “complementary,” “complement,” or “complementary nucleic acid sequence” refers to the nucleic acid strand that is related to the base sequence in another nucleic acid strand by the Watson-Crick base-pairing rules. In general, two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G/U or U/G basepairs.

The term “hybrid” refers to a double-stranded nucleic acid molecule formed by hydrogen bonding between complementary nucleotides. The term “hybridize” refers to the process by which single strands of nucleic acid sequences form double-helical segments through hydrogen bonding between complementary nucleotides.

The term “support” or “surface” refers to a porous or non-porous water insoluble material. The support can have any one of a number of shapes, such as strip, plate, disk, rod, particle, including bead, and the like. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; flat glass whose surface has been chemically activated to support binding or synthesis of polynucleotides, glass available as Bioglass, ceramics, metals, and the like. Natural or synthetic assemblies such as liposomes, phospholipid vesicles, and cells can also be employed. Binding of oligonucleotides to a support or surface may be accomplished by well-known techniques, commonly available in the literature. See, for example, A. C. Pease, et al., Proc. Nat. Acad. Sci. USA 91:5022-5026 (1994).

The term “related sequences” refers to sequences having a variation in nucleotides such as in a “mutation,” for example, single nucleotide polymorphisms. In general, the variations occur from individual to individual. The mutation may be a change in the sequence of nucleotides of normally conserved nucleic acid sequence to resulting in the formation of a mutant as differentiated from the normal (unaltered) or wild-type sequence. Point mutations (i.e. mutations at a single base position) can be divided into two general classes, namely, base-pair substitutions and frameshift mutations. The latter entail the insertion or deletion of a nucleotide pair. Mutations that insert or delete multiple base pairs are also possible; these can leave the translation frame unshifted, permanently shifted, or shifted over a short stretch of sequence. A difference of a single nucleotide can be significant so to change the phenotype from normality to abnormality as in the case of, for example, sickle cell anemia.

The phrase “amplification of nucleic acids or polynucleotides” refers to any method that results in the formation of one or more copies of a nucleic acid or polynucleotide molecule (exponential amplification) or in the formation of one or more copies of only the complement of a nucleic acid or polynucleotide molecule (linear amplification).

The phrase “potential of an oligonucleotide to hybridize” refers to the combination of duplex formation rate and duplex dissociation rate that determines the amount of duplex nucleic acid hybrid that will form under a given set of experimental conditions in a given amount of time.

An “array” includes any one-, two- or three-dimensional arrangement of addressable regions bearing a particular polynucleotide associated with that region. An array is addressable in that it has multiple regions of different polynucleotide sequences such that a region or feature or spot of the array at a particular predetermined location or address on the array can detect a particular target molecule or class of target molecules although a feature may incidentally detect non-target molecules of that feature. An “array assembly” on the surface of a support refers to one or more arrays disposed along a surface of an individual support and separated by inter-array areas. The arrays can be designed for testing against any type of sample, whether a trial sample, a reference sample, a combination of the foregoing, or a known mixture of polynucleotides (in which case the arrays may be composed of features carrying unknown sequences to be evaluated). The surface of the support may carry at least one, two, four, or at least ten, arrays. Depending upon intended use, any or all of the arrays may be the same or different from one another and each may contain multiple spots or features of polynucleotides including oligonucleotides. A typical array may contain more than ten, more than one hundred, more than one thousand or ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. Each feature, or element, within the molecular array is defined to be a small, regularly shaped region of the surface of the substrate. The features are arranged in a predetermined manner. Any of a variety of geometries of arrays on a support may be used. As mentioned above, an individual support may contain a single array or multiple arrays. Features of the array may be arranged in rectilinear rows and columns. This is particularly attractive for single arrays on a support. When multiple arrays are present, such arrays can be arranged, for example, in a sequence of curvilinear rows across the substrate surface (for instance, a sequence of concentric circles or semi-circles of spots), and the like. Similarly, the pattern of features may be varied from the rectilinear rows and columns of spots to include, for example, a sequence of curvilinear rows across the support surface (for example, a sequence of concentric circles or semi-circles of spots), and the like. The configuration of the arrays and their features may be selected according to manufacturing, handling, and use considerations.

The term “parameter” refers to a factor that provides information about the hybridization of an oligonucleotide with a target nucleotide sequence. Generally, the factor is one that is predictive of the ability of an oligonucleotide to hybridize with a target nucleotide sequence. Such factors include composition factors, thermodynamic factors, chemosynthetic efficiencies, kinetic factors, and the like. The term also includes empirical factors such as the experimentally testing the hybridization of oligonucleotides with target nucleotide sequences.

The phrase “parameter predictive of the ability to hybridize” refers to a parameter calculated from a set of oligonucleotide sequences wherein the parameter positively correlates with observed hybridization efficiencies of those sequences. The parameter is, therefore, predictive of the ability of those sequences to hybridize. “Positive correlation” can be rigorously defined in statistical terms. The correlation coefficient ρ_{x,y} of two experimentally measured discreet quantities x and y (N values in each set) is defined as

$${ρ}_{x , y} = \frac{Covariance ( x , y )}{\sqrt{Variance ( x ) Variance ( y )}} ,$$

where the Covariance (x,y) is defined by

$$Covariance ( x , y ) = \frac{1}{N} {\sum }_{j = 1}^{N} ( {x}_{j} - {μ}_{x} ) ( {y}_{l} - {μ}_{y} ) .$$

The quantities μₓ and μ_{y} are the averages of the quantities x and y, while the variances are simply the squares of the standard deviations (defined below). The correlation coefficient is a dimensionless (unitless) quantity between −1 and 1. A correlation coefficient of 1 or −1 indicates that x and y have a linear relationship with a positive or negative slope, respectively. A correlation coefficient of zero indicates no relationship; for example, two sets of random numbers will yield a correlation coefficient near zero. Intermediate correlation coefficients indicate intermediate degrees of relatedness between two sets of numbers. The correlation coefficient is a good statistical measure of the degree to which one set of numbers predicts a second set of numbers.

The phrase “composition factor” refers to a numerical factor based solely on the composition or sequence of an oligonucleotide without involving additional parameters, such as experimentally measured nearest-neighbor thermodynamic parameters. For instance, the fraction (G+C), given by the formula

$${f}_{GC} = \frac{{n}_{G} + {n}_{C}}{{n}_{G} + {n}_{C} + {n}_{A} + {n}_{T or U}} ,$$

where n_{G}, n_{C}, n_{A} and n_{T or U} are the numbers of G, C, A and T (or U) bases in an oligonucleotide, is an example of a composition factor. Examples of composition factors, by way of illustration and not limitation, are mole fraction (G+C), percent (G+C), sequence complexity, sequence information content, frequency of occurrence of specific oligonucleotide sequences in a sequence database and so forth.

The phrase “thermodynamic factor” refers to numerical factors that predict the behavior of an oligonucleotide in some process that has reached equilibrium. For instance, the free energy of duplex formation between an oligonucleotide and its complement is a thermodynamic factor. Thermodynamic factors for systems that can be subdivided into constituent parts are often estimated by summing contributions from the constituent parts. Such an approach is used to calculate the thermodynamic properties of oligonucleotides.

Examples of thermodynamic factors, byway of illustration and not limitation, are predicted duplex melting temperature, predicted enthalpy of duplex formation, predicted entropy of duplex formation, free energy of duplex formation, predicted melting temperature of the most stable intramolecular structure of the oligonucleotide or its complement, predicted enthalpy of the most stable intramolecular structure of the oligonucleotide or its complement, predicted entropy of the most stable intramolecular structure of the oligonucleotide or its complement, predicted free energy of the most stable intramolecular structure of the oligonucleotide or its complement, predicted melting temperature of the most stable hairpin structure of the oligonucleotide or its complement, predicted enthalpy of the most stable hairpin structure of the oligonucleotide or its complement, predicted entropy of the most stable hairpin structure of the oligonucleotide or its complement, predicted free energy of the most stable hairpin structure of the oligonucleotide or its complement, thermodynamic partition function for intramolecular structure of the oligonucleotide or its complement and the like.

Chemosynthetic efficiency—oligonucleotides and nucleotide sequences may both be made by sequential polymerization of the constituent nucleotides. In such circumstances the individual addition steps are not perfect; they instead proceed with some fractional efficiency that is less than unity. This may vary as a function of position in the sequence. Therefore, what is really produced is a family of molecules that consists of the desired molecule plus many truncated sequences. These “failure sequences” affect the observed efficiency of hybridization between an oligonucleotide and its complementary target. Examples of chemosynthetic efficiency factors, by way of illustration and not limitation, are coupling efficiencies, overall efficiencies of the synthesis of a target nucleotide sequence or an oligonucleotide probe, and so forth.

The phrase “kinetic factor” refers to numerical factors that predict the rate at which an oligonucleotide hybridizes to its complementary sequence or the rate at which the hybridized sequence dissociates from its complement is called kinetic factors. Examples of kinetic factors are steric factors calculated via molecular modeling, rate constants calculated via molecular dynamics simulations, associative rate constants, dissociative rate constants, enthalpies of activation, entropies of activation, free energies of activation, and the like.

The phrase “evaluating a parameter” refers to determination of the numerical value of a numerical descriptor of a property of an oligonucleotide sequence by means of a formula, algorithm or look-up table.

The term “filter” refers to a mathematical rule or formula that divides a set of numbers into two subsets. Generally, one subset is retained for further analysis while the other is discarded. In the context of the current invention, an example by way of illustration and not limitation is the statement “The predicted self structure free energy must be greater than or equal to −0.4 kcal/mole,” which can be used as a filter for oligonucleotide sequences.

The phrase “filter set” refers to a set of rules or formulae that successively winnow a set of numbers by identifying and discarding subsets that do not meet specific criteria. In the context of the current invention, an example by way of illustration and not limitation is the compound statement. “The predicted self structure free energy must be greater than or equal to −0.4 kcal/mole and the predicted RNA/DNA heteroduplex melting temperature must lie between 60° C. and 85° C.,” which can be used as a filter set for oligonucleotide sequences.

The phrase “predicted duplex melting temperature” refers to the temperature at which an oligonucleotide mixed with a hybridizable nucleotide sequence is predicted to form a duplex structure (double-helix hybrid) with 50% of the hybridizable sequence. At higher temperatures, the amount of duplex is less than 50%; at lower temperatures, the amount of duplex is greater than 50%. The melting temperature Tₘ(° C.) is calculated from the enthalpy (ΔH), entropy (ΔS) and C, the concentration of the most abundant duplex component (for hybridization arrays, the soluble hybridization target), using the equation

$${T}_{in} = \frac{Δ H}{Δ S + R ln C} - 273.15 ,$$

where R is the gas constant, 1.987 cal/(mole-°K). For longer sequences (>100 nucleotides), Tₘ can also be estimated from the mole fraction (G+C),_{X}_{^{G+C}}, using the equation Tₘ=81.5+41.0_{X}_{_{G+C}}.

“Predicted enthalpy, entropy and free energy of duplex formation” refers to the enthalpy (ΔH), entropy and free energy (ΔG) are thermodynamic state functions, related by the equation ΔG=ΔH−TΔS,

where T is the temperature in °K. In practice, the enthalpy and entropy are predicted via a thermodynamic model of duplex formation (the “nearest neighbor” model which is explained in more detail below), and used to calculate the free energy and melting temperature.

“Predicted free energy of the most stable intramolecular structure of an oligonucleotide or its complement” refers to single-stranded DNA and RNA molecules that contain self-complementary sequences that can form intramolecular secondary structures. Many such structures are possible for a given sequence; two are of particular interest. The first is the lowest energy “hairpin” structure (formed by folding a sequence back on itself with a connecting loop at least 3 nucleotides long). The second is the lowest energy structure that can be formed by including more complex topologies, such as “bulge loops” (unpaired duplexes between two regions of base-paired duplex) and cloverleaf structures, where 3 base-paired stretches meet at a triple-junction. A good example of a complex secondary structure is the structure of a t-RNA molecule, an example of which is Yeast t-RNA^{Ala} (see, for example, A. L. Lehninger, et al., Principles of Biochemistry, 2^{nd} Ed. (Worth Publishers, New York, N.Y., 1993)).

For either type of structure, a value of the free energy of that structure can be calculated, relative to the unpaired strand, by means of a thermodynamic model similar to that used to calculate the free energy of a base-paired duplex structure. Again, the free energy ΔG is calculated from the enthalpy ΔH and the entropy ΔS at a given absolute temperature T via the equation ΔG=ΔH−TΔS.

However, in this case there is the added difficulty that the lowest energy structure must be found. For a simple hairpin structure, this optimization can be performed via a relatively simple search algorithm. For more complex structures (such as a cloverleaf) a dynamic programming algorithm, such as that implemented in the program MFOLD, must be used.

“Coupling efficiencies” refers to chemosynthetic efficiencies, which are called coupling efficiencies when the synthetic scheme involves successive attachment of different monomers to a growing oligomer; a good example is oligonucleotide synthesis via phosphoramidite coupling chemistry.

The phrase “statistical sampling of a cluster” refers to extraction of a subset of oligonucleotides from a cluster of oligonucleotides based upon some statistical measure, such as rank by oligonucleotide starting position in the sequence complementary to the target sequence.

“Melting temperature corrected for salt concentration” refers to polynucleotide duplex melting temperatures, which are calculated with the assumption that the concentration of sodium ion, Na⁺, is 1 M. Melting temperatures T′ₘ calculated for duplexes formed at different salt concentrations are corrected via the semi-empirical equation T′ₘ([Na⁺])=Tₘ+16.6 log([Na⁺]).

The phrase “poorly correlated” may be understood as follows: if it is not possible to perform a “good” prediction, as defined via statistics, of one set of numbers from another set of numbers using a simple linear model, then the two sets of numbers are said to be poorly correlated.

“Computer program” refers to a written set of instructions that symbolically instructs an appropriately configured computer to execute an algorithm that will yield desired outputs from some set of inputs. The instructions may be written in one or several standard programming languages, such as C, C⁺¹, Visual BASIC, FORTRAN or the like. Alternatively, the instructions may be written by imposing a template onto a general-purpose numerical analysis program, such as a spreadsheet.

The phase “hybridization efficiency” refers to the productivity of a hybridization reaction, measured as either the absolute or relative yield of oligonucleotide probe/polynucleotide target duplex formed under a given set of conditions in a given amount of time.

Homologous or substantially identical polynucleotides—in general, two polynucleotide sequences that are identical or can each hybridize to the same polynucleotide sequence are homologous. The two sequences are homologous or substantially identical where the sequences each have at least 90%, preferably 100%, of the same or analogous base sequence where thymine (T) and uracil (U) are considered the same. Thus, the ribonucleotides A, U, C and G are taken as analogous to the deoxynucleotides dA, dT, dC, and dG, respectively. Homologous sequences can both be DNA or one can be DNA and the other RNA.

Complementary—Two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G=U or U=G basepairs.

Support or surface—a porous or non-porous water insoluble material. The surface can have any one of a number of shapes, such as strip, plate, disk, rod, particle, including bead, and the like. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. Natural or synthetic assemblies such as liposomes, phospholipid vesicles, and cells can also be employed.

Binding of oligonucleotides to a support or surface may be accomplished by well-known techniques, commonly available in the literature. See, for example, A. C. Pease, et al., Proc. Nat. Acad. Sci. USA, 91:5022-5026 (1994).

Label—a member of a signal producing system. Usually the label is part of a target nucleotide sequence or an oligonucleotide probe, either being conjugated thereto or otherwise bound thereto or associated therewith. The label is capable of being detected directly or indirectly. Labels include (i) reporter molecules that can be detected directly by virtue of generating a signal, (ii) specific binding pair members that may be detected indirectly by subsequent binding to a cognate that contains a reporter molecule, (iii) oligonucleotide primers that can provide a template for amplification or ligation or (iv) a specific polynucleotide sequence or recognition sequence that can act as a ligand such as for a repressor protein, wherein in the latter two instances the oligonucleotide primer or repressor protein will have, or be capable of having, a reporter molecule. In general, any reporter molecule that is detectable can be used.

Ancillary Materials—various ancillary materials will frequently be employed in the methods and assays utilizing oligonucleotide probes designed in accordance with the present invention. For example, buffers and salts will normally be present in an assay medium, as well as stabilizers for the assay medium and the assay components. Frequently, in addition to these additives, proteins may be included, such as albumins, organic solvents such as formamide, quaternary ammonium salts, polycations such as spermine, surfactants, particularly non-ionic surfactants, binding enhancers, e.g., polyalkylene glycols, or the like.

### Specific Embodiments

As mentioned above one embodiment of the present invention is a method for predicting the potential of an oligonucleotide to hybridize to a target nucleotide sequence. A predetermined number of predictor oligonucleotides is identified. The length of the predictor oligonucleotides may be the same or different. The predictor oligonucleotides are unique in that no two of the oligonucleotides are identical. The predictor oligonucleotides are chosen to sample the entire length of a nucleotide sequence that is hybridizable with the target nucleotide sequence. The actual number of predictor oligonucleotides is generally determined by the length of the nucleotide sequence and the desired result. The number of predictor oligonucleotides should be sufficient to achieve a consensus behavior. In other words, the predictor oligonucleotide sequences should be sufficiently numerous that several possible probes overlap or fall within a given region that is expected to yield acceptable hybridization efficiency. Since the location of these regions is not known beforehand, the best strategy is to equally space the predictor probe sequences along the sequence that is hybridizable to the target sequence. Since in the present invention it is desired to prepare hybridization oligonucleotides that are at least about 30 nucleotides in length, usually, at least about 40 nucleotides in length, more usually, at least about 60 nucleotides in length and may be up to about 100 nucleotides in length, regions of acceptable hybridization efficiency are generally on the order of 20 to 25 nucleotides in length. Accordingly, a practical strategy is to space the starting nucleotides of the predictor oligonucleotide sequences no more than about 3 base pairs apart. If computation time needed to calculate the predictive parameters is not an issue, then the best strategy is to space the starting nucleotides one nucleotide apart. As part of the present invention predictor oligonucleotides that are clustered along a region of the nucleotide sequence are determined.

Preferably, a set of overlapping predictor sequences is chosen. To this end, the subsequences are chosen so that there is overlap of at least one nucleotide from one predictor oligonucleotide to the next. More preferably, the overlap is two or more nucleotides. Most preferably, the oligonucleotides are spaced one nucleotide apart and the predetermined number is L−N+1 predictor oligonucleotides where L is the length of the nucleotide sequence and N is the length of the predictor oligonucleotides. In the latter situation, the predictor oligonucleotides are of identical length N. Thus, a set of overlapping predictor oligonucleotides is a set of predictor oligonucleotides that are subsequences derived from some master sequence by subdividing that sequence in such a way that each subsequence contains either the start or end of at least one other subsequence in the set.

An example of the above for purposes of illustration and not limitation is presented by the sequence ATGGACTTAGCATTCG (SEQ ID NO: 1), from which the following set of overlapping predictor oligonucleotides can be identified:

| | |
|---|---|
| ATGGACTTAGCA | (SEQ ID NO:2) |
| TGGACTTAGCAT | (SEQ ID NO:3) |
| GGACTTAGCATT | (SEQ ID NO:4) |
| GACTTAGCATTC | (SEQ ID NO:5) |
| ACTTAGCATTCG | (SEQ ID NO:6) |

In this example the overlapping predictor oligonucleotides are spaced one nucleotide apart. In other words, there is overlap of all but one nucleotide from one predictor oligonucleotide to the next. In the example above, the original nucleotide sequence is 16 nucleotides long (L=16). The length of each of the overlapping predictor oligonucleotides is 12 nucleotides long (N=12) and there are L−N+1=5 oligonucleotides.

The length of the predictor oligonucleotides may be the same or different and may vary depending on the length of the nucleotide sequence. The length of the predictor oligonucleotides for use in the present invention is dependent on the methods used to determine the filter settings used to screen the predictor oligonucleotides, the average GC content of the target organism (higher CC contents dictate shorter predictor probe lengths), details of the array fabrication process (e.g. whether or not the probes are spaced away from the array surface via non-hybridizing polynucleotide “stilts”) and the results of objective, empirical optimization of the predictor probe length. Usually, the length of the predictor oligonucleotides is from about 20 to 30 nucleotides, more usually, from about 23 to 27 nucleotides.

In the next step of the method, at least one parameter that is independently predictive of the ability of each of the predictor oligonucleotides of the set to hybridize to the target nucleotide sequence is determined and evaluated for each of the above oligonucleotides. Examples of such a parameter, by way of illustration and not limitation, is a parameter selected from the group consisting of composition factors, thermodynamic factors, chemosynthetic efficiencies, kinetic factors and mathematical combinations of these quantities.

The determination of a parameter may be carried out by known methods. For example, melting temperature of the oligonucleotide:target duplex may be determined using the nearest neighbor method and parameters appropriate for the nucleotide acids involved. For DNA/DNA parameters, see J. SantaLucia Jr., et al., (1996) Biochemistry, 35:3555. For RNA/DNA parameters, see N. Sugimoto, et al., (1995) Biochemistry, 34:11211. Briefly, these methods are based on the observation that the thermodynamics of a nucleic acid duplex can be modeled as the sum of a term arising from the entire duplex and a set of terms arising from overlapping pairs of nucleotides (“nearest neighbor” model). For a discussion of the nearest neighbor see J. SantaLucia Jr., et al., (1996) Biochemistry, supra, and N. Sugimoto, et al., (1995) Biochemistry, supra. For example, the enthalpy ΔH of the duplex formed by the sequence

| | |
|---|---|
| ATGGACTTAGCA | (SEQ ID NO:2) |

and its perfect complement can be approximated by the equation

$$Δ H ≅ {H}_{nnt} + {H}_{AT} + {H}_{IG} + {H}_{GG} + {H}_{GA} + {H}_{AC} + {H}_{CT} + {H}_{TT} + {H}_{IA} + {H}_{AG} + {H}_{GC} + {H}_{CA} .$$

In the above equation, the term Hᵢₙᵢₜ is the initiation enthalpy for the entire duplex, while the terms H_{AΓ}, . . . , H_{CA} are the so-called “nearest neighbor” enthalpies. Similar equations can be written for the entropy, for the corresponding quantities for RNA homoduplexes, or for DNA:RNA heteroduplexes. The free energy can then be calculated from the enthalpy, entropy and absolute temperature, as described previously.

Predicted free energy of the most stable intramolecular structure of an oligonucleotide (ΔG_{MFOLD}) may be determined using the nucleic acid folding algorithm MFOLD and parameters appropriate for the oligonucleotide, e.g., DNA or RNA. For MFOLD, see J. A. Jaeger, et al., (1989), supra. For DNA folding parameters, see J. SantaLucia Jr., et al., (1996), supra. Briefly, these methods operate in two steps. First, a map of all possible compatible intramolecular base pairs is made. Second, the global minimum of the free energy of the various possible base pairing configurations is found, using the nearest neighbor model to estimate the enthalpy and entropy, the user input temperature to complete the calculation of free energy, and a dynamic programming algorithm to find the global minimum. The algorithm is computationally intensive; calculation times scale as the third power of the sequence length.

The following Table 1 summarizes groups of parameters that are independently predictive of the ability of each of the predictor oligonucleotides to hybridize to the target nucleotide sequence together with a reference to methods for their determination. Parameters within a given group are known or expected to be strongly correlated to one another, while parameters in different groups are known or expected to be poorly correlated with one another.

TABLE 1

| Group | Parameter | Reference |
|---|---|---|
| I | duplex enthalpy, ΔH | Santa Lucia et al, 1996; Sugimoto et al, 1995 |
| | duplex entropy, ΔS | Santa Lucia et al, 1996; Sugimoto et al, 1995 |
| | duplex free energy, ΔG | ΔG = ΔH − TΔS (see text) |
| | melting temperature, Tₘ | |
| | mole fraction (or percent) G + C | self-explanatory |
| | subsequence duplex enthalpy | Santa Lucia et al, 1996; Sugimoto et al, 1995 |
| | subsequence duplex entropy | Santa Lucia et al, 1996; Sugimoto et al, 1995 |
| | subsequence duplex free energy | ΔG = ΔH − TΔS (see text) |
| | subsequence duplex Tₘ | |
| | subsequence duplex mole fraction (or | self-explanatory |
| | percent) G + C | |
| II | intramolecular enthalpy, ΔH_{MFOLD} | Jaeger at al., 1989; Santa Lucia et al., 1996 |
| | intramolecular entropy, ΔS_{MFOLD} | Jaeger at al., 1989; Santa Lucia et al., 1996 |
| | intramolecular free energy, ΔG_{MFOLD} | ΔG = ΔH − TΔS (see text) |
| | hairpin enthalpy, ΔHₕₐᵢᵣₚᵢₙ | Jaeger at al., 1989; Santa Lucia et al., 1996 |
| | hairpin entropy, ΔSₕₐᵢᵣₚᵢₙ | Jaeger at al., 1989; Santa Lucia et al., 1996 |
| | hairpin free energy, ΔGₕₐᵢᵣₚᵢₙ | ΔG = ΔH − TΔS (see text) |
| | intramolecular partition function | |
| III | sequence complexity | Altschul et al, 1994 |
| | sequence information content | Altschnl et al, 1994 |
| IV | steric factors | |
| | molecular dynamic simulation | |
| | enthalpy, entropy & free energy of | measured experimentally |
| | activation | |
| | association & dissociation rates | measured experimentally |
| V | oligonucleotide chemosynthetic | measured experimentally |
| | efficiencies | |
| VI | target synthetic efficiencies | measured experimentally |

As mentioned above, one parameter involves the experimental determination of the hybridization of a predetermined number of unique oligonucleotides with target sequences. In general, this approach involves synthesizing an array comprising probes to the target sequences. Oligonucleotide probes of a predetermined length of about 20 to about 30 nucleotides are synthesized and allowed to react with the target sequences. The ability of the oligonucleotide probes to hybridize to the target sequences is evaluated and the oligonucleotide probes are ranked on the basis of this evaluation to identify predictor oligonucleotides. In a particular example, 25-mer probes may be empirically tested using an array of target sequences. The array is designed to contain 25-mers spanning the target sequences. The unique oligonucleotide probes are designed by generating 25-mer probes from the sequences of the target sequences, such as, for example, spacing by 3 (i.e., probes starting at bases 3, 6, 9,12, etc.). Arrays generated with this design are hybridized individually with an array of target sequences. Analysis of probe performance may be based, for example, on signal intensity from a suitable label as a function of probe position. From this analysis, regions of the target sequences where 25-mer probes exhibit high signals may be identified and the oligonucleotide probes may be ranked to determine predictor oligonucleotides. The method of the invention may be applied as discussed above to generate oligonucleotide probes of increased length over the predictor oligonucleotides.

In a next step of the present method, a subset of predictor oligonucleotides within the predetermined number of predictor oligonucleotides is identified based on the above evaluation of the parameter. A number of mathematical approaches may be followed to sort the predictor oligonucleotides based on a parameter. In one approach a cut-off value is established. The cut-off value is adjustable and can be optimized relative to one or more training data sets. This is done by first establishing some metric for how well a cutoff value is performing; for example, one might use the normalized signal observed for each oligonucleotide in the training set. Once such a metric is established, the cutoff value can be numerically optimized to maximize the value of that metric, using optimization algorithms well known to the art. Alternatively, the cutoff value can be estimated using graphical methods, by graphing the value of the metric as a function of one or more parameters, and then establishing cutoff values that bracket the region of the graph where the chosen metric exceeds some chosen threshold value. In essence, the cut off values are chosen so that the rule set used yields training data that maximizes the inclusion of oligonucleotides that exhibit good hybridization efficiency and minimizes the inclusion of oligonucleotides that exhibit poor hybridization efficiency.

Once the cut-off value is selected, a subset of predictor oligonucleotides having parameter values greater than or equal to the cut-off value is identified. This refers to the inclusion of predictor oligonucleotides in a subset based on whether the value of a predictive parameter satisfies an inequality.

Examples of identifying a subset of oligonucleotides by establishing cut-off values for predictive parameters are as follows: for melting temperature an inequality might be 60° C.≦Tₘ; for predicted free energy an inequality, preferably, might be

$$Δ {G}_{MFOLD} \geq - 0.4 \frac{kcal}{mole} .$$

In a variation of the above, both a maximum and a minimum cut-off value may be selected. A subset of oligonucleotides is identified whose values fall within the maximum and minimum values, i.e., values greater than or equal to the minimum cut-off value and less than or equal to the maximum cut-off value. An example of this approach for melting temperature might be the inequality 60° C.≦Tₘ≦85° C.

With regard to cut-off values for Tₘ the lower limit is preferably Tₘ=T_{hyb}, more preferably, Tₘ=T_{hyb}+5° C., with no upper limit. From a practical point of view the upper limit should not be so great so that the target exhibits so much self-structure that predictive value is lost. The upper cut-off is important when the sequence region under consideration is unusually rich in G and C. With regard to ΔG_{MFOLD} the cut-off value is usually greater than or equal to −1.0 kcal/mole. As mentioned above, the cut-off values preferably are determined from real data through experimental observations.

In another approach the parameter values may be converted into dimensionless numbers. The parameter value is converted into a dimensionless number by determining a dimensionless score for each parameter resulting in a distribution of scores having a mean value of zero and a standard deviation of one. The dimensionless score is a number that is used to rank some object (such as an oligonucleotide) to which that score relates. A score that has no units (i.e., a pure number) is called a dimensionless score.

In the simplest approach a dimensionless score is obtained by having units in the numerator and denominator that cancel each other. This may be achieved by dividing the quantity to be converted into a dimensionless parameter by some characteristic or “natural” value of that parameter. For example, DNA duplex melting temperatures could be converted into a dimensionless parameter by dividing by the average of the melting temperatures of all duplexes of the same length. In this case, the parameter would measure the fraction by which a particular Tₘ differed from the average, with a value of 1 indicating no difference. Dimensionless parameters can be made even simpler by expressing them as differences from some reference value of that parameter. In the example above, this could be achieved by subtracting 1 from all values, yielding a parameter that measures only the difference from average behavior, with a value of 0 indicating average behavior.

In one approach the following equations are used for converting the values of said parameters into dimensionless numbers:

$${s}_{i , x} = \frac{{x}_{i} - ⟨ x ⟩}{{σ}_{\{ x \}}} ,$$

where s_{i,x} is the dimensionless score derived from parameter x calculated for oligonucleotide i, x, is the value of parameter x calculated for oligonucleotide i, <x> is the average of parameter x calculated for all of the predictor oligonucleotides under consideration for a given nucleotide sequence target, and σ_{{x}} is the standard deviation of parameter x calculated for all of the predictor oligonucleotides under consideration for a given nucleotide sequence target, and is given by the equation

$${σ}_{\{ x \}} = \sqrt{\frac{{\sum }_{j = 1}^{M} {( {x}_{i} - ⟨ x ⟩ )}^{2}}{M - 1}} ,$$

where M is the number of predictor oligonucleotides. The resulting distribution of scores, {s} has a mean value of zero and a standard deviation of one. These properties can be important for a combination of the scores discussed below.

The use of a dimensionless number approach may further include calculating a combination score S, by evaluating a weighted average of the individual values of the dimensionless scores s_{i,x} by the equation:

$${S}_{i} = {\sum }_{⟨ x ⟩}^{} {q}_{i} {s}_{i , x} ,$$

where qₓ is the weight assigned to the score derived from parameter x, the individual values of qₓ are always greater than zero, and the sum of the weights qₓ is unity.

In another variation of the above approach, the method of calculation of the composite parameter is optimized based on the correlation of the individual composite scores to real data, as explained more fully below.

In one approach the calculation of the composite score further involves determining a moving window-averaged combination score <Sᵢ> for the ith probe by the equation:

$$⟨ {S}_{i} ⟩ = \frac{1}{w} {\sum }_{j = i - \frac{w - 1}{2}}^{1 + \frac{w - 1}{2}} {S}_{i} ,$$

w=an odd integer,

where w is the length of the window for averaging (i.e., w nucleotides long), and then applying a cutoff filter to the value of <Sᵢ>. This procedure results in smoothing (smoothing procedure) by turning each score into a consensus metric for a set of w adjacent oligonucleotide probes. The score, referred to as the “smoothed score,” is essentially continuous rather than a few discrete values. The value of the smoothed score is strongly influenced by clustering of scores with high or low values; window averaging therefore provides a measurement of cluster size.

An advantage of the dimensionless score approach to the probe prediction algorithm is that it is easy to objectively optimize. In one approach to training the algorithm, optimization of the weights qₓ above may be performed by varying the values of the weights so that the correlation coefficient ρ_{{<Si>},{Vi}} between the set of window-averaged combination scores {<S_{i>} and a set of calibration experimental measurements {V}_{1}} is maximized. The correlation coefficient ρ_{{<Si>},{Vi}} is calculated from the equation

$${ρ}_{\{ < {S}_{i} > \} , \{ {V}_{i} \}} = ( \frac{1}{M} ) \frac{Covariance ( ⟨ S ⟩ , V )}{{σ}_{\{ < {S}_{i} > \} } {σ}_{\{ {V}_{i} \}}} ,$$

where M is the number of window averaged, combination dimensionless scores and the number of corresponding measurements, the covariance is as defined earlier (see earlier equations) and σ_{{<Si>}} and σ_{{Vt}} are the standard deviations of {<Sᵢ>} and {Vₜ}, as defined previously. An example of this approach is shown in Example 2, below.

In another approach the parameter is derived from one or more factors by mathematical transformation of the factors. This involves the calculation of a new predictive parameter from one or more existing predictive parameters, by means of an equation. For instance, the equilibrium constant Kₒₚₑₙ for formation of an oligonucleotide with no intramolecular structure from its structured form can be calculated from the intramolecular structure free energy ΔG_{MFOLD}, using the equation:

$${K}_{open} = exp ( \frac{Δ {G}_{MFOLD}}{RT} ) .$$

In a next step of the method, predictor oligonucleotides in the subset are then identified that are clustered along a region of the nucleotide sequence that is hybridizable to the target nucleotide sequence. For example, consider a set of overlapping predictor oligonucleotides identified by dividing a nucleotide sequence into subsequences. A subset of the predictor oligonucleotides is obtained as described above. In general, this subset is obtained by applying a rule that rejects some members of the set. For the remaining members of the set, namely, the subset, there will be some average number of nucleotides in the nucleotide sequence between the first nucleotides of adjacent remaining subsequences. If, for some sub-region of the nucleotide sequence, the average number of nucleotides in the nucleotide sequence between the first nucleotides of adjacent remaining subsequences is less than the average for the entire nucleotide sequence, then the predictor oligonucleotides are clustered or in other words are “in clusters.” The smaller the average number of nucleotides between the first nucleotides of adjacent oligonucleotides, the stronger is the clustering. The strongest clustering occurs when there are no intervening nucleotides between adjacent starting nucleotides. In this case, the predictor oligonucleotides are said to be contiguous and may be referred to as contiguous sequence elements or “contigs.”

Accordingly, in this step predictor oligonucleotides are sorted based on length of contiguous sequence elements. Predictor oligonucleotides in the subset determined above are identified that are contiguous along a region of the input nucleic acid sequence. The length of each contig that is equal to the number of oligonucleotides in each contig, namely, oligonucleotides from the above step whose complement begin at positions m+1, m+2, . . . , m+k in the target sequence, form a contig of length k. Contigs can be identified and contig length can be calculated using, for example, a Visual Basic® module that can be incorporated into a Microsoft® Excel workbook or a similar module incorporated into a database, such as Microsoft® Access or an Oracle® database application.

Cluster size can be defined in several ways. For contiguous clusters, the size is simply the number of adjacent predictor oligonucleotides in the cluster. Again, this may also be referred to as contiguous sequence elements. The number may also be referred to as “contig length”. For example, consider the nucleotide sequence discussed above, namely, ATGGACTTAGCATTCG (SEQ ID NO:1) and the identified set of overlapping oligonucleotides

| | |
|---|---|
| ATGGACTTAGCA | (SEQ ID NO:2) |
| TGGACTTAGCAT | (SEQ ID NO:3) |
| GGACTTAGCATT | (SEQ ID NO:4) |
| GACTTAGCATTC | (SEQ ID NO:5) |
| ACTTAGCATTCG | (SEQ ID NO:6) |

Suppose that, after calculation and evaluation of the predictive parameters, four nucleotides remain:

| | | |
|---|---|---|
| ATGGACTTACCA | (SEQ ID NO:2) | &Rectversolid; |
| TGGACTTAGCAT | (SEQ ID NO:3) | &Rectversolid; contig |
| GGACTTAGCATT | (SEQ ID NO:4) | &Rectversolid; |
| ACTTAGCATTCG | (SEQ ID NO:6) | &Rectversolid; single oligonu- |
| | | cleotide |

A “contig” corresponding to the length of three of the oligonucleotides of the subset is present together with a single oligonucleotide. The contig length is 3 oligonucleotides.

Alternatively, cluster size at some position in the sequence hybridizable or complementary to the target sequence may be defined as the number of predictor oligonucleotides whose center nucleotides fall inside a region of length M centered about the position in question, divided by M. This definition of clustering allows small gaps in clusters. In the example used above for contiguous clusters, if M was 10, then the cluster size would step through the values 0/10, . . . , 0/10, 1/10, 2/10, 3/10, 3/10, 4/10, 4/10, 4/10, 4/10, 4/10, 3/10, 2/10, 1/10, 1/10, 0/10 as the center of the window of length 10 passed through the cluster. In each fraction, the numerator is the number of oligonucleotide sequences that have satisfied the filter set and whose central nucleotides are within a window 10 nucleotides long, centered about the nucleotide under consideration. The denominator (10) is simply the window length.

Another alternative is to define the size of a cluster at some position in the sequence hybridizable or complementary to the target sequence as the number of predictor oligonucleotide sequences overlapping that position. This definition is equivalent to the last definition with M set equal to the oligonucleotide probe length and omission of the division by M.

Finally, cluster size can be approximated at each position in a nucleotide sequence by dividing the sequence into predictor oligonucleotides, evaluating a numerical score for each predictor oligonucleotide, and then averaging the scores in the neighborhood of each position by means of a moving window average as described above. Window averaging has the effect of reinforcing clusters of high or low values around a particular position, while canceling varying values about that position. The window average, therefore, provides a score that is sensitive to both the hybridization potential of a given predictor oligonucleotide and the hybridization potentials of its neighbors.

In a next step of the present method, the clusters of predictor oligonucleotides are ranked. Generally, this ranking is based on the number of predictor oligonucleotides in each of the clusters or contigs, sizes of the clusters or values of a window-averaged score. The highest ranking is given to the cluster with greatest number of predictor oligonucleotides and the clusters are ranked in descending order according to the number of predictor oligonucleotides in the clusters. We have found that hybridization oligonucleotides determined from clusters of largest size exhibit high specificity and sensitivity toward a target nucleotide sequence. Accordingly, in the present method a determination is first made of the length or number of nucleotides that is desired for the hybridization oligonucleotide to be selected based on the selected cluster. In this way a hybridization oligonucleotide of predetermined length is selected. The desired or predetermined length of the hybridization oligonucleotide is dependent on a number of considerations including the concentration of the labeled sample to be hybridized to the array, the number of distinguishable polynucleotide species present in the sample (sample complexity), the chemical efficiency of probe synthesis, the absence or presence of steric constraints to hybridization, the stringency of the hybridization process, the volume of the hybridization sample, the length of time over which hybridization is performed and the sensitivity of the detection technology used to quantitate the degree of hybridization.

In the present method each hybridization oligonucleotide may be obtained or selected by a process that begins with the central nucleotide of each contig and adds nucleotides in one direction (3′ or 5′) or from both directions (3′ and 5′) from the central nucleotide. When added to the central nucleotide in both directions, the nucleotides may be added equally (symmetrically) to each end or they may be added unequally, i.e., a greater number of nucleotides may be added in one direction than in the other direction (non-symmetrically). The added nucleotides may correspond only to the nucleotides within the contig or may extend beyond the nucleotides of the contig in one or both directions. The hybridization oligonucleotide may comprise at least a portion of the nucleotide sequence of the contig, and in one embodiment may comprise at least the entire sequence of the contig. In another embodiment the hybridization oligonucleotide may comprise a portion of the sequence of the contig plus a sequence of nucleotides, which is not in the contig and which corresponds to a sequence of nucleotides of the nucleotide sequence where such sequence of nucleotides is adjacent the contig. In another embodiment the hybridization oligonucleotide comprises at least one half of the nucleotide sequence including a central nucleotide of the contig plus a sequence of nucleotides that is not in the contig and that corresponds to a sequence of nucleotides of the nucleotide sequence where such sequence of nucleotides is adjacent the contig.

The particular process in accordance with the present invention that is utilized depends upon the type of array being designed and its manner of synthesis. The following approaches are by way of example and not limitation. In one example, oligonucleotide arrays are synthesized by in situ synthesis techniques with the 3′-end of the oligonucleotide attached to the support. In this situation, addition of nucleotides to the central nucleotide in the 3′ direction may be the approach of choice. The reason for this is that the 10 or so bases closest to the support of an in situ-synthesized array are known to be compromised by the proximity of the surface and by crowding from short “failure sequences” (sequences which were started but terminated due to crowding or side reactions), which are found in abundance near the attachment surface. Since the shorter sequence that was originally designed was expected to be a particularly efficient probe, it makes the most sense to move this sequence as far from the support surface as possible.

Another example is an oligonucleotide array synthesized in situ where the 5′-end of each oligonucleotide is attached to the array support. In this example, addition to the central nucleotide in accordance with the present invention in the 5′ direction may be most appropriate. The reason for this is essentially the same as that given above for the reverse situation.

Another example is an oligonucleotide array prepared by deposition techniques using, for example, photo-cross-link attachment of the oligonucleotide to the support of the array. In this situation, there is no preferred probe end. Accordingly, symmetric addition to the central nucleotide in accordance with the present invention may be the process of choice.

Another example is an oligonucleotide array synthesized by in situ methods where the 3′-end or 5′-end of the oligonucleotide is attached to the array support and extension is 30 or more bases in length. In this situation, either symmetric addition or addition at the surface-attached end will move the shorter sequence that was originally designed to a height of 15 or more bases above the attachment surface. This height is sufficient to eliminate the effects of surface proximity and “failure sequences” on most currently used surfaces. In this case, the advantages of extending the surface-anchored end of the oligonucleotide must be weighed against the decrease in oligonucleotide yield with increasing length of the oligonucleotide. For example, if the average step yield of a phosphoramidite-based in situ oligonucleotide synthesis is 99%, then the final yield for synthesis of a 60-mer probe is 100×(0.99)⁶⁰=55%; this will be the final probe yield for asymmetric synthesis. The corresponding symmetric addition of a 25-mer test probe with a 15-base extension at the attached probe end will complete synthesis of the test probe at base 15+25=40. The yield at that point will be 100×(0.99)⁴⁰=67%. Thus, if achieving maximum synthetic yield of the test sequence is deemed a more important design goal than achieving maximum displacement of the test sequence from the surface, then symmetric addition is a better design strategy than addition at the attached end in this particular example.

Various ways may be employed to introduce the reagents for producing an array of polynucleotides on the surface of a support such as a glass support. Such methods are known in the art. One such method is discussed in U.S. Pat. No. 5,744,305 (Fodor, et al.) and involves solid phase chemistry, photolabile protecting groups and photolithography. Binary masking techniques are employed in one embodiment of the above. Arrays are fabricated in situ, adding one base pair at a time to a primer site. Photolithography is used to uncover sites, which are then exposed and reacted with one of the four base pair phosphoramidites. In photolithography the surface is first coated with a light-sensitive resist, exposed through a mask and the predetermined area is revealed by dissolving away the exposed or the unexposed resist and, subsequently, a surface layer. A separate mask is usually made for each predetermined area, which may involve one for each base pair in the length of the probe.

Another in situ method employs inkjet printing technology to dispense the appropriate phosphoramidite reagents and other reagents onto individual sites on a surface of a support. Oligonucleotides are synthesized on a surface of a substrate in situ using phosphoramidite chemistry. Solutions containing nucleotide monomers and other reagents as necessary such as an activator, e.g., tetrazole, are applied to the surface of a support by means of thermal ink-jet technology. Individual droplets of reagents are applied to reactive areas on the surface using, for example, a thermal ink-jet type nozzle. The surface of the support may have an alkyl bromide trichlorosilane coating to which is attached polyethylene glycol to provide terminal hydroxyl groups. These hydroxyl groups provide for linking to a terminal primary amine group on a monomeric reagent. Excess of non-reacted chemical on the surface is washed away in a subsequent step. For example, see U.S. Pat. No. 5,700,637 and PCT WO 95/25116 and PCT application WO 89/10977.

Another approach for fabricating an array of biopolymers on a substrate using a biopolymer or biomonomer fluid and using a fluid dispensing head is described in U.S. Pat. No. 6,242,266 (Schleifer, et al.). The head has at least one jet that can dispense droplets onto a surface of a support. The jet includes a chamber with an orifice and an ejector, which, when activated, causes a droplet to be ejected from the orifice. Multiple droplets of the biopolymer or biomonomer fluid are dispensed from the head orifice so as to form an array of droplets on the surface of the substrate.

In another embodiment (U.S. Pat. No. 6,232,072) (Fisher) a method of, and apparatus for, fabricating a biopolymer array is disclosed. Droplets of fluid carrying the biopolymer or biomonomer are deposited onto a front side of a transparent substrate. Light is directed through the substrate from the front side, back through a substrate back side and a first set of deposited droplets on the first side to an image sensor.

An example of another method for chemical array fabrication is described in U.S. Pat. No. 6,180,351 (Cattell). The method includes receiving from a remote station information on a layout of the array and an associated first identifier. A local identifier is generated corresponding to the first identifier and associated array. The local identifier is shorter in length than the corresponding first identifier. The addressable array is fabricated on the substrate in accordance with the received layout information.

Other methods for synthesizing arrays of oligonucleotides on a surface include those disclosed by Gamble, et al., WO97/44134; Gamble, et al., WO98/10858;

Baldeschwieler, et al., WO95/25116; Brown, et al., U.S. Pat. No. 5,807,522; and the like.

The dimensions of the support may vary depending on the nature of the support and the nature of the chemical reactions to be performed. For example, the support may be one on which is synthesized a single array of chemical compounds that are biopolymers. In this regard the support is usually about 1.5 to about 5 inches in length and about 0.5 to about 3 inches in width. The support is usually about 0.1 to about 5 mm, more usually, about 0.5 to about 2 mm, in thickness. A standard size microscope slide is usually about 3 inches in length and 1 inch in width. Alternatively, multiple arrays of chemical compounds may be synthesized on the support, which is then diced, i.e., cut, into single array supports. In this alternative approach the support is usually about 5 to about 8 inches in length and about 5 to about 8 inches in width so that the support may be diced into multiple single array supports having the aforementioned dimensions. The thickness of the support is the same as that described above. In a specific embodiment by way of illustration and not limitation, a wafer that is 6⅝ inches by 6 inches is employed and diced into one inch by 3 inch slides.

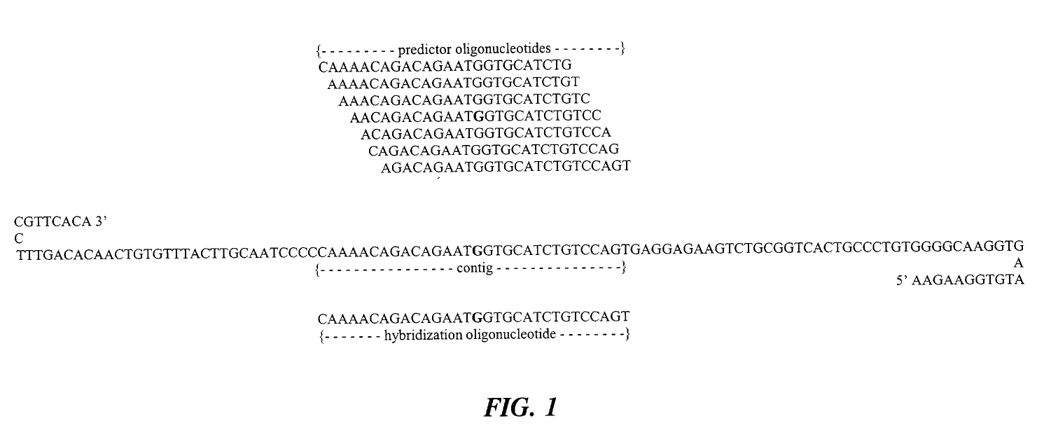

As mentioned above, in one approach in accordance with the present invention, the nucleotides are added to the central nucleotide in a symmetrical manner at each end, i.e., upstream or downstream of the central nucleotide or 3′ and 5′ of the central nucleotide. The nucleotides correspond to the nucleotides adjacent the central nucleotide in the contig region of the nucleotide sequence on which the predictor oligonucleotides form clusters of predictor oligonucleotides. An example of this process by way of illustration and not limitation is set forth in FIG. 1.

Referring to FIG. 1, a nucleotide sequence (SEQ ID NO:7) (depicted in wrap around fashion due to space constraints), which is 122 nucleotides in length and complementary to a target nucleotide sequence. Seven predictor oligonucleotides of length 25 nucleotides each (SEQ ID NO: 8 to SEQ ID NO:14, respectively) are clustered to form a contig (SEQ ID NO:15) having 31 nucleotides of the nucleotide sequence. The central nucleotide of the contig is nucleotide G (shown in bold), which becomes the central nucleotide of a hybridization oligonucleotide in accordance with the present invention. In this example the predetermined length of the hybridization oligonucleotide is 31 nucleotides. Therefore, in accordance with the present invention, nucleotides are added equally to each end where the nucleotides correspond to the nucleotides of the contig along the nucleotide sequence. Accordingly, nucleotide T is added downstream and nucleotide G is added upstream to give TGG. Next, nucleotide A is added downstream and nucleotide T is added upstream to give ATGGT. This process is continued until the desired length is obtained for hybridization oligonucleotide: CAAAACAGACAGAATGGTGCATCTGTCCAGT (SEQ ID NO:15) (31 nucleotides).

Additional hybridization oligonucleotides are chosen using the remaining clusters in descending order of rank. In other words the next largest of the ranked clusters is selected. As above, a hybridization oligonucleotide that has as its central nucleotide the central nucleotide of a region of the nucleotide sequence that corresponds to the cluster otherwise referred to as the contig. The remaining nucleotides of the hybridization oligonucleotide are added in correspondence to the nucleotides in the nucleotide sequence that extend equally in both directions from the central nucleotide, until a predetermined length of the hybridization oligonucleotide is obtained. In the above example, the predetermined length for the hybridization oligonucleotide was 31. The process is repeated by selecting the next largest cluster and so forth until a desired or predetermined number of hybridization oligonucleotides of length 31 nucleotides are obtained, one from each cluster.

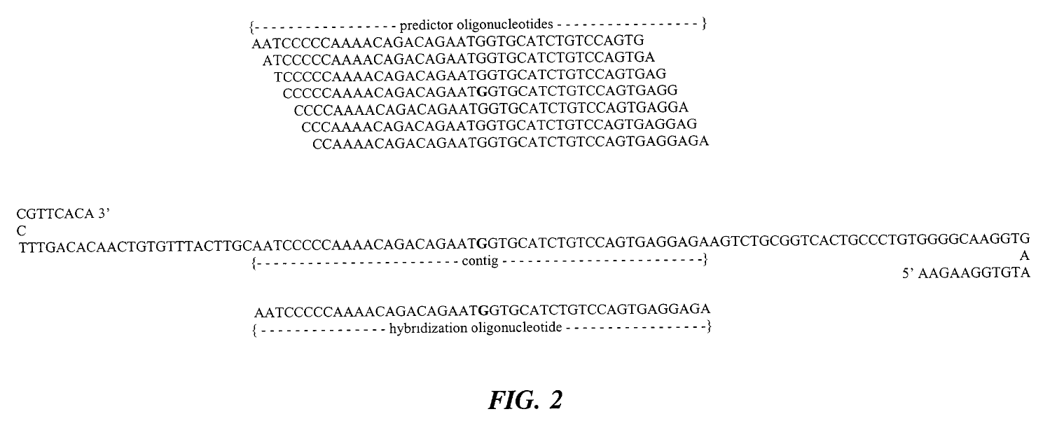

In the above example the number of nucleotides in the contig region of the nucleotide sequence corresponds to the predetermined number of nucleotides desired in the hybridization oligonucleotide. For hybridization oligonucleotides of predetermined length that is greater than 31, several approaches may be employed. For example, referring to FIG. 2, for a hybridization oligonucleotide that is 45 nucleotides in length, oligonucleotides that are 39 nucleotides in length may be employed. The method of the invention is repeated using these oligonucleotides. Clusters or contigs are identified and ranked as discussed above. Nucleotides are added symmetrically to the central nucleotide of the contig. The nucleotides correspond to the nucleotides of the contig along the nucleotide sequence. In this example, the contig along the nucleotide sequence is 45 nucleotides. Accordingly, the hybridization oligonucleotide obtained in this example spans the entire length of the contig. Accordingly, for a contig that contains 7 predictor oligonucleotides (SEQ ID NO:16 to SEQ ID NO:22) of length 39 nucleotides, a hybridization oligonucleotide that is 45 nucleotides in length is obtained, namely, AATCCCCCAAAACAGACAGAATGGTGCATCTGTCCAGTGAGGAGA (SEQ ID NO:23).

Hybridization oligonucleotides having a predetermined length that is longer may be obtained using shorter predictor oligonucleotides in several ways. In one approach the duplex melting temperature parameter may be relaxed as the predetermined length for the hybridization oligonucleotide is increased. The rationale for this approach is an observed property of oligonucleotide duplexes: as sequence length increases, duplex melting temperature becomes independent of sequence (it depends only on fraction (G+C)). In this way clusters are obtained with an increased number of predictor oligonucleotides thereby expanding the contig region corresponding to the nucleotide sequence. Typically, the duplex melting temperature parameter is relaxed about 0.0° C. to about 1.0° C., usually, relaxed about 0.25° C. to about 0.75° C., for each increase about 1 nucleotides in the predetermined length of the hybridization oligonucleotide.

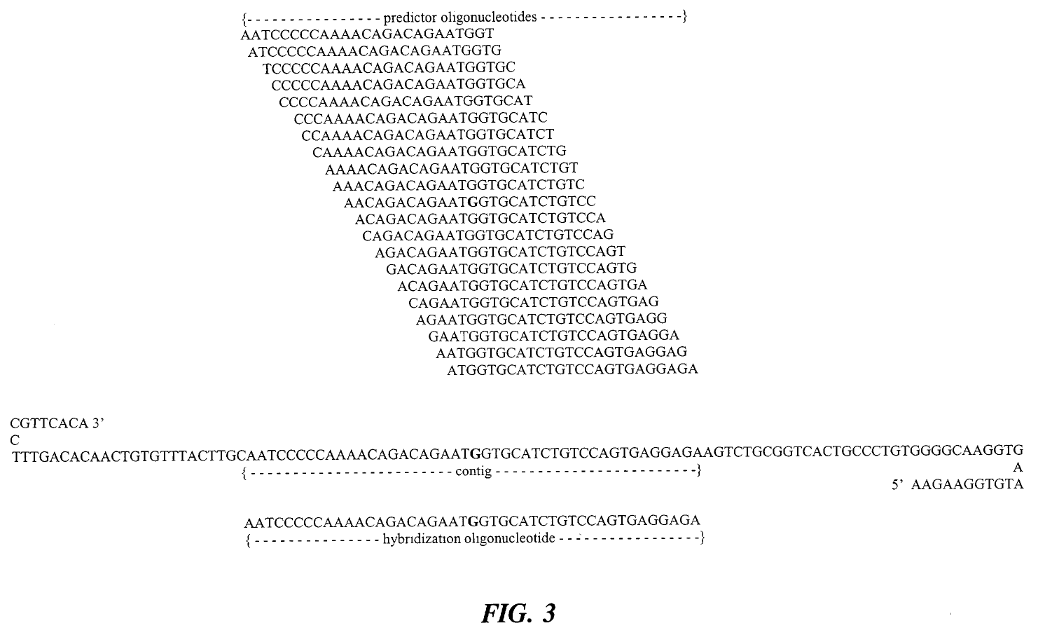

An example of the above is described next with reference to FIG. 3. Predictor oligonucleotides that are 25 nucleotides in length are employed to obtain a hybridization oligonucleotide having a predetermined length of 45 nucleotides. The approach is similar to that discussed above for the example of FIG. 1. However, the duplex melting temperature parameter for the predictor oligonucleotide:nucleotide sequence duplex is relaxed by 10.0° C. The method of the invention is repeated using these oligonucleotides. Clusters or contigs are identified and ranked as discussed above. As a result of the relaxation of the duplex melting temperature parameter, the cluster of predictor oligonucleotides along the nucleotide sequence is expanded to 21. Nucleotides are added symmetrically to the central nucleotide of the contig. The nucleotides correspond to the nucleotides of the “contig” along the nucleotide sequence. In this example, the contig along the nucleotide sequence is 45 nucleotides. The hybridization oligonucleotide obtained in this example spans the entire length of the contig. Accordingly, for a contig that contains 19 predictor oligonucleotides (SEQ ID NO:24 to SEQ ID NO:30, SEQ ID NO:8 to SEQ ID NO:14, SEQ ID NO: 31 to SEQ ID NO:37) of length 25 nucleotides, a hybridization oligonucleotide that is 45 nucleotides in length is obtained, namely, AATCCCCCAAAACAGACAGAATGGTGCATCTGTCCAGTGAGGAGA (SEQ ID NO:23).

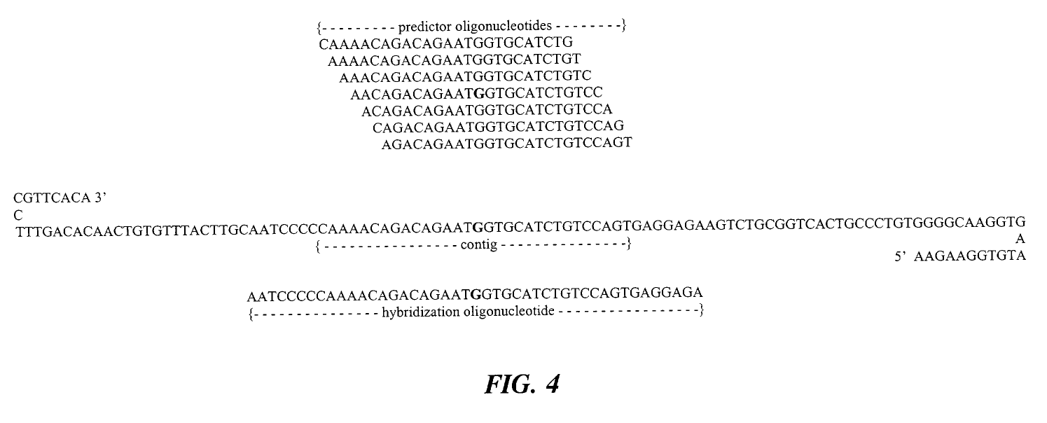

In another approach using shorter predictor oligonucleotides in obtaining hybridization oligonucleotides of longer length, the hybridization oligonucleotide may be selected by adding nucleotides symmetrically to a central nucleotide where some of the added nucleotides are outside of, or extend beyond, the “contig” along the nucleotide sequence. Referring to FIG. 4, for example, a nucleotide sequence (SEQ ID NO:7) is shown that is 122 nucleotides in length and complementary to a target nucleotide sequence. Seven predictor oligonucleotides (SEQ ID NO:8 to SEQ ID NO:14) are clustered to form a contig having 31 nucleotides corresponding to 31 nucleotides of the nucleotide sequence. The predictor oligonucleotides in the cluster are each 25 nucleotides in length. The central nucleotide of the contig is nucleotide G (shown in bold), which becomes the central nucleotide of a hybridization oligonucleotide in accordance with the present invention. In this example the predetermined length of the hybridization oligonucleotide is 45 nucleotides. As discussed above, nucleotides are added equally to each end where the nucleotides correspond to the nucleotides of the contig along the nucleotide sequence. Accordingly, nucleotide T is added downstream and nucleotide G is added upstream to give TGG. Next, nucleotide A is added downstream and nucleotide T is added upstream to give ATGGT. An oligonucleotide that is 31 nucleotides in length corresponds to the length of the contig. However, additional nucleotides are added to obtain the desired hybridization oligonucleotide that is 45 nucleotides in length. Accordingly, the symmetrical addition of nucleotides is continued until the desired length is obtained for the hybridization oligonucleotide, namely, AATCCCCCAAAACAGACAGAATGGTGCATCTGTCCAGTGAGGAGA (SEQ ID NO:23) (45 nucleotides). As can be seen, this length is longer than the length of the contig, which is 31 nucleotides in length.

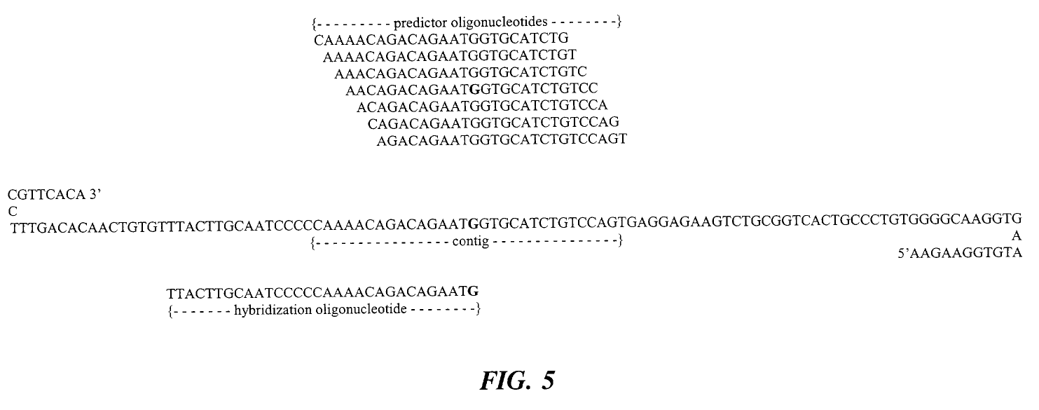

In another embodiment of the invention, the nucleotides are added to the central nucleotide in a non-symmetrical manner. Thus, the nucleotides may be added solely in one direction or unequally in both directions. In one approach in accordance with this embodiment, the nucleotides are added in one direction, i.e., upstream or downstream of the central nucleotide or 3′ and 5′ of the central nucleotide. The nucleotides correspond to the nucleotides adjacent the central nucleotide in the contig region of the nucleotide sequence on which the predictor oligonucleotides form clusters of predictor oligonucleotides. An example of this process by way of illustration and not limitation is set forth in FIG. 5

Referring to FIG. 5, a nucleotide sequence (SEQ ID NO:7), which is 122 nucleotides in length and complementary to a target nucleotide sequence. Seven predictor oligonucleotides of length 25 nucleotides each (SEQ ID NO: 8 to SEQ ID NO:14, respectively) are clustered to form a contig (SEQ ID NO:15) having 31 nucleotides of the nucleotide sequence. The central nucleotide of the contig is nucleotide G (shown in bold), which becomes the central nucleotide of a hybridization oligonucleotide in accordance with the present invention. In this example the predetermined length of the hybridization oligonucleotide is 31 nucleotides. Therefore, in accordance with the present invention, nucleotides are added to the central nucleotide G in a 3′ direction. This process is continued until the desired length is obtained for hybridization oligonucleotide TTACTTGCAATCCCCCAAAACAGACAGAATG (SEQ ID NO:38) (31 nucleotides).

As above, additional hybridization oligonucleotides are chosen using the remaining clusters in descending order of rank. In other words the next largest of the ranked clusters is selected. As above, a hybridization oligonucleotide that has as its central nucleotide the central nucleotide of a region of the nucleotide sequence that corresponds to the cluster otherwise referred to as the contig. The remaining nucleotides of the hybridization oligonucleotide are added in correspondence to the nucleotides in the nucleotide sequence that extend in the 3′ direction from the central nucleotide, until a predetermined length of the hybridization oligonucleotide is obtained. In the above example, the predetermined length for the hybridization oligonucleotide was 31. The process is repeated by selecting the next largest cluster and so forth until a desired or predetermined number of hybridization oligonucleotides of length 31 nucleotides are obtained, one from each cluster.

With regard to the discussion above, one aspect of the present invention is the use of three or more parameters in the determination of clusters and the number of nucleotides that may be added outside the contig. Predictor oligonucleotides may be evaluated using parameters that are particularly important for shorter oligonucleotides and using a third parameter that is particularly important for longer oligonucleotides. Contigs may be determined based on the evaluation of predictor oligonucleotides in view of the first two parameters. These contigs should be within or overlap the evaluation using the third parameter. From the contig information, a hybridization oligonucleotide may be constructed as discussed above. Nucleotides may be added symmetrically from the central nucleotide to obtain the hybridization oligonucleotide. However, the number of nucleotides added outside the contig should not be so great that the third parameter values for the resulting hybridization oligonucleotide fall outside of the cut-off value for the third parameter.

This may be explained more fully using the following parameters as examples. Two parameters that are important for shorter oligonucleotide probes, melting temperature Tₘ and the self-structure free energy ΔG_{MFOLD}, are calculated for each of the potential predictor oligonucleotide probe: target nucleotide sequence complexes. The values are obtained from a user-written function that calculates DNA/RNA heteroduplex thermodynamic parameters (see N. Sugimoto, et al., Biochemistry, 34:11211 (1995)) and a modified version of the program MFOLD that estimates the free energy of the most stable intramolecular structure of a single stranded DNA molecule (see J. A. Jaeger, et al., (1989), supra), respectively.

Next, the oligonucleotide sequences are filtered on the basis of Tₘ. A high and low cut-off value may be selected, for example, 60° C.≦Tₘ≦85° C. Thus, oligonucleotides having Tₘ values falling within the above range are retained. Those outside the range are discarded. Next, the predictor oligonucleotide sequences remaining after the above exercise are filtered on the basis of ΔG_{MFOLD} and are retained if the value is greater than, for example, −0.4. Those oligonucleotides with a ΔG_{MFOLD} less than, for example, −0.4 are discarded. Clusters of retained oligonucleotides are identified and ranked based on cluster size. A hybridization oligonucleotide is obtained as described above from each cluster according to descending cluster rank. These hybridization oligonucleotides are evaluated experimentally with emphasis on a third parameter, namely, homology sequence count. To this end each predictor sequence is evaluated for homology to the sequences in a representative expressed sequence database for the organism in question, using a standard homology search algorithm, such as BLAST (Altschul, S. F., W. Gish, et al. (1990). “Basic local alignment search tool.” J Mol Biol 215(3): 403-10). A representative expressed sequence database is a database containing the sequences of expressed genes, with duplicate entries removed. Examples include the database of all annotated yeast open reading frames and the human Unigene unique database. The number of homologies found that exceed some stringency threshold (for example, yielding a BLAST “E value” less than 0.3) are counted. Predictor probes are then filtered according to the number of homologies counted. For example, all probes yielding 2 or more counted homologies might be discarded, while probes yielding 0 or 1 counted homologies would be retained.

The aforementioned methods of the present invention are preferably carried out at least in part with the aid of a computer. For example, an IBM® compatible personal computer (PC) may be utilized. The computer is driven by software specific to the methods described herein.

The preferred computer hardware capable of assisting in the operation of the methods in accordance with the present invention involves a system with at least the following specifications: Pentium® processor or better with a clock speed of at least 100 MHz, at least 32 megabytes of random access memory (RAM) and at least 80 megabytes of virtual memory, running under either the Windows 95 or Windows NT 4.0 operating system (or successor thereof).

As mentioned above, software that may be used to carry out the methods may be, for example, Microsoft Excel or Microsoft Access, suitably extended via user-written functions and templates, and linked when necessary to stand-alone programs that calculate specific parameters (e.g., MFOLD for intramolecular thermodynamic parameters). Examples of software programs used in assisting in conducting the present methods may be written, preferably, in Visual BASIC, FORTRAN and C⁺⁺, as exemplified below in the Examples. It should be understood that the above computer information and the software used herein are by way of example and not limitation. The present methods may be adapted to other computers and software. Other languages that may be used include, for example, PASCAL, PERL or assembly language.

In one aspect of the present method as discussed above, at least two parameters are determined wherein the parameters are poorly correlated with respect to one another. The reason for requiring that the different parameters chosen be poorly correlated with one another is that an additional parameter that is strongly correlated to the original parameter brings no additional information to the prediction process. The correlation to the original parameter is a strong indication that both parameters represent the same physical property of the system. Another way of stating this is that correlated parameters are linearly dependent on one another, while poorly correlated parameters are linearly independent of one another. In practice, the absolute value of the correlation coefficient between any two parameters should be less than 0.5, more preferably, less than 0.25, and, most preferably, as close to zero as possible.

In one preferred approach, instead of Tₘ, for each predictor oligonucleotide:nucleotide sequence duplex, the difference between the predicted duplex melting temperature corrected for salt concentration and the temperature of hybridization of each of the predictor oligonucleotides with the target nucleotide sequence is determined.

This window summing procedure converts the score for the passed value for each predictor oligonucleotide into a consensus metric for a set of w adjacent probes. A “consensus metric” is a measurement that distills a number of values into one consensus value. In this case, the consensus value is calculated by simply summing the individual values. The window summing procedure therefore evaluates a property similar to the contig length metric discussed above. However, the summed score has the advantage of allowing for a few probes within a cluster to have not passed their individual probe score limits. We have found that this allows more observed hybridization peaks to be predicted.

It may be desired in some circumstances to combine the results of multiple algorithm versions. This operation is referred to herein as “tiling”. This may be explained more fully as follows. Tiling generally involves joining the predicted predictor oligonucleotide probe sets identified by multiple algorithm versions. In the context of the present invention, tiling multiple algorithm versions involves forming the unions or intersections of multiple sets of predictions. These predictions may arise from different embodiments of the present invention. Alternatively, the different sets of predictions may arise from the same embodiment, but different filter sets. The different filter sets may additionally be restricted to different combinations of parameter values. For instance, one filter set might be used when the predicted duplex melting temperature Tₘ is greater than or equal to some value, while another might be used when Tₘ is below that value.

The hybridization oligonucleotide probes find use in polynucleotide assays particularly where the assays involve oligonucleotide arrays. For a discussion of oligonucleotide arrays, see, e.g., U.S. Pat. No. 5,700,637 (E. Southern) and U.S. Pat. No. 5,667,667 (E. Southern), the relevant disclosures of which are incorporated herein by reference.

Another aspect of the present invention is a computer-based method for predicting the potential of an oligonucleotide to hybridize to a target nucleotide sequence. A predetermined number of unique predictor oligonucleotides within a nucleotide sequence that is hybridizable with the target nucleotide sequence is identified under computer control. The predictor oligonucleotides are chosen to sample the entire length of the nucleotide sequence. A value is determined and evaluated under computer control for each of the predictor oligonucleotides for at least one parameter that is independently predictive of the ability of each of the predictor oligonucleotides to hybridize to the target nucleotide sequence. The parameter values are stored. Based on the evaluating of the parameter, a subset of oligonucleotides within the predetermined number of unique predetermined oligonucleotides is identified under computer control from the stored parameter values. Then, oligonucleotides in the subset that are clustered along a region of the nucleotide sequence that is hybridizable to the target nucleotide sequence are identified under computer control. The clusters are ranked under computer control in order of number of oligonucleotides in each cluster from greatest number to least number of oligonucleotides. A hybridization oligonucleotide is selected under computer control for each cluster, in descending order of cluster rank. The selected hybridization oligonucleotide has as its central nucleotide the central nucleotide of a region of the nucleotide sequence that corresponds to the cluster. Under computer control, the remaining nucleotides of the hybridization oligonucleotide are added, in correspondence to the nucleotides in the nucleotide sequence that extend in one or both directions from the central nucleotide, until a hybridization oligonucleotide of predetermined length is obtained and a predetermined number of hybridization oligonucleotides are obtained, one from each cluster. The higher the rank of the cluster, the higher is the hybridization potential of the hybridization oligonucleotide for the target nucleotide sequence.

A computer program is utilized to carry out the above method steps. The computer program provides for (i) input of a target-hybridizable or target-complementary nucleotide sequence, (ii) efficient algorithms for computation of predictor oligonucleotide sequences and their associated predictive parameters, (iii) efficient, versatile mechanisms for filtering sets of predictor oligonucleotide sequences based on parameter values, (iv) mechanisms for computation of the size of clusters of oligonucleotide sequences that pass multiple filters, mechanisms for identifying a central nucleotide of a cluster and adding nucleotides symmetrically to the central nucleotide, based on the nucleotide sequence, to form a hybridization oligonucleotide, and (v) mechanisms for outputting the final predictions of the method of the present invention in a versatile, machine-readable or human-readable form.

Another embodiment of the present invention is a computer system for conducting a method for predicting the potential of a hybridization oligonucleotide to hybridize to a target nucleotide sequence. The system comprises input means for introducing a target nucleotide sequence into the computer system. The input means may permit manual input of the target nucleotide sequence. The input means may also be a database or a standard format file such as GenBank. The system further comprises means for determining a number of unique predictor oligonucleotide sequences that are within a nucleotide sequence that is hybridizable with the target nucleotide sequence. The predictor oligonucleotide sequences are chosen to sample the entire length of the nucleotide sequence. Suitable means is a computer program or software, which also provides memory means for storing the oligonucleotide sequences. The system also comprises memory means for storing the information regarding the predictor oligonucleotide sequences.

The system also comprises means for controlling the computer system to carry out a determination and evaluation for each of the oligonucleotide sequences a value for at least one parameter that is independently predictive of the ability of each of the oligonucleotide sequences to hybridize to the target nucleotide sequence. Suitable means is a computer program or software such as, for example, Microsoft® Excel spreadsheet, Microsoft® Access relational database or the like, which also provides memory means for storing the parameter values. The system further comprises means for storing the parameter values. Suitable means is a computer program or software, which also provides memory means for storing the subset of oligonucleotides. The system also comprises means for controlling the computer to carry out an identification from the stored parameter values a subset of oligonucleotide sequences within the number of unique oligonucleotide sequences based on the evaluation of the parameter. Suitable means is a computer program or software, which also provides memory means for storing the oligonucleotide sequences in the subset. The system also includes means for storing the subset of oligonucleotides. Suitable means is a computer program or software, which also provides memory means for storing the oligonucleotide sequences in the subset.

The system further includes means for controlling the computer to carry out an identification of oligonucleotide sequences in the subset that are clustered along a region of the nucleotide sequence that is hybridizable to the target nucleotide sequence. Suitable means is a computer program or software. The system also includes means for storing the oligonucleotide sequences in the subset. Suitable means is a computer program or software, which also provides memory means for storing the oligonucleotide sequences in the subset. The computer also comprises means for outputting data relating to the oligonucleotide sequences in the subset. The system further comprises means for ranking the clusters in order of number of oligonucleotides in each cluster from greatest number to least number of oligonucleotides. Suitable means is a computer program or software. The system also includes means for selecting for each cluster, in descending order of cluster rank, a hybridization oligonucleotide that has as its central nucleotide the central nucleotide of a region of the nucleotide sequence that corresponds to the cluster, the remaining nucleotides of the hybridization oligonucleotide being added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster. Suitable means is a computer program or software. The computer system also comprises means for outputting data relating to the hybridization oligonucleotides in a versatile, machine-readable or human-readable form. Such means may be machine readable or human readable and may be software that communicates with a printer, electronic mail, another computer program, and the like. One particularly attractive feature of the present invention is that the outputting means may communicate directly with software that is part of an oligonucleotide synthesizer. In this way the results of the method of the present invention may be used directly to provide instruction for the synthesis of the desired oligonucleotides.

Another advantage of the present invention is that it may be used to provide efficient hybridization oligonucleotides for each of multiple target sequences. Thus, very large arrays may be constructed and tested with minimal synthesis of oligonucleotides.

In another embodiment of the present invention, matched specificity, or cross-hybridization, control probes for each potential hybridization oligonucleotide are designed and the performance of the hybridization oligonucleotides is evaluated experimentally using the specificity control probes. Thus, in this embodiment of the invention a set of hybridization oligonucleotides that are specific for a target nucleotide sequence and are of length greater than about 30 nucleotides is selected according to the above methods. In addition, a set comprising a minimum number of cross-hybridization control oligonucleotide probes for use, if necessary, in analyzing a target nucleotide sequence is also selected. The target-specific hybridization oligonucleotide probes may be employed in a method for analyzing a target nucleotide sequence. Using the present invention one can select longer hybridization oligonucleotide probes that are sufficiently specific for a target nucleotide sequence so that cross-hybridization of such probes with interfering sequences that may be present in a sample does not significantly affect the ability to detect the target sequence. In this circumstance the target-specific hybridization oligonucleotide probes may be employed in the method of analysis without using cross-hybridization probes. However, if one is not able to achieve the above, then, the present invention provides for identifying a minimum number of cross-hybridization oligonucleotide probes that may be used in conjunction with the target-specific hybridization oligonucleotide probes to reduce the impact of such cross-hybridization events to an acceptable level by providing means for measuring a correction factor that partially or completely cancels the signal generated by cross-hybridization events. It is further within the purview of the present invention to use the identified cross-hybridization probes to adjust the signal obtained so that the method of analysis will result in an accurate measurement of the amount of the target nucleic acid sequence. Usually, the signal is adjusted so as to reduce its level by at most about 50%, more usually, by about 5% to 20%.

In the present method a cross-hybridization oligonucleotide probe is identified based on a candidate longer length, target-specific hybridization oligonucleotide probe for the target nucleotide sequence determined as described above. The cross-hybridization oligonucleotide probe measures a signal that can be used to estimate the extent of the occurrence of a cross-hybridization event between the target-specific hybridization oligonucleotide probe and an interfering sequence having a predetermined probability.

The cross-hybridization oligonucleotide probe is identified by a process comprising deletion of at least one nucleotide from the hybridization oligonucleotide. In the present invention the deletion(s) are evenly spaced with respect to the nucleotides of the hybridization oligonucleotide. The number of deletions generally is based on the length of the cross-hybridization oligonucleotide probe. Usually, there is one deletion of a nucleotide from the hybridization oligonucleotide for every 15 to 25 nucleotides of the hybridization oligonucleotide.

For example, consider the hybridization oligonucleotide identified as described above and depicted in FIG. 1. The hybridization oligonucleotide is 31 nucleotides in length: CAAAACAGACAGAATGGTGCATCTGTCCAGTG (SEQ ID NO:15).

According to the present invention one nucleotide is deleted from the above and the deletion is evenly spaced. Thus, nucleotide 16 (shown in bold above and coincidentally corresponding to the central nucleotide), namely, G, is deleted to obtain a cross-hybridization probe CAAAACAGACAGAATGTGCATCTGTCCAGTG (SEQ ID NO:39) that is 30 nucleotides long.

In another example, consider the hybridization oligonucleotide identified as described above and depicted in FIG. 3. The hybridization oligonucleotide, namely, AATCCCCCAAAACAGACAGAATGGTGCATCTGTCCAGTGAGGAGA (SEQ ID NO:23) is 45 nucleotides in length. According to the present invention two nucleotides are deleted from the above hybridization oligonucleotide to identify a cross-hybridization probe. The deletions are evenly spaced. Therefore, nucleotides 15 and 30, namely, G and T (shown in bold) are deleted to obtain a cross-hybridization probe, AATCCCCCAAAACAACAGAATGGTGCATCGTCCAGTGAGGAGA (SEQ ID NO:40) that is 43 nucleotides in length.

Of course, there may be the situation where the hybridization oligonucleotide is of a length that the nucleotide deletion cannot be exactly evenly spaced. Therefore, it is within the purview of the present invention that the nucleotide deletion be as close to evenly spaced as possible. In any event “evenly” as used in this application allows for the spacing to vary from an exact even spacing by not more than 5 nucleotides, usually, not more than 2 nucleotides, although exact even spacing could be used where possible. For example, consider a hybridization oligonucleotide that is 52 nucleotides in length such as, for example,

TTGCAATCCCCCAAAACAGACAGAATGGTGCATCTGTCCAGTGAGGAGAAGT (SEQ ID NO:41). To obtain a cross-hybridization probe from this hybridization oligonucleotide, nucleotides 17 and 34 may be deleted or nucleotides 16 and 34 or nucleotides 17 and 33.

In a particular embodiment the hybridization oligonucleotide is 60 nucleotides in length and the deletions are made at nucleotides 15, 30 and 45 of the hybridization oligonucleotide to yield a cross-hybridization probe. In another particular embodiment the hybridization oligonucleotide is 60 nucleotides in length and the deletions are made at nucleotides 12, 24, 36 and 48 of the hybridization oligonucleotide to yield a cross-hybridization probe.

Another embodiment of the present invention is a method for selecting a set of target-specific oligonucleotide probes of at least about 20 nucleotides in length for use in analyzing a target nucleotide sequence. A cross-hybridization oligonucleotide probe is identified based on the target nucleic acid sequence by a process comprising deletion of at least one nucleotide from a single hybridization oligonucleotide. The deletion(s) are evenly spaced with respect to the nucleotides of the hybridization oligonucleotide and the number of deletions is based on the length of the cross-hybridization oligonucleotide probe. Cross-hybridization results are determined employing the cross-hybridization oligonucleotide probe and target-specific oligonucleotide probe. Target-specific oligonucleotide probes are selected for the set based on the cross-hybridization results.

In general, the extent of the occurrence of a cross-hybridization event is one that is predicted to be highly probable or most probable. Cross-hybridization probes are identified based on a certain threshold level, which may be adjusted on a case by case basis to assure that a sufficient number of probes are included for consideration. Then, probes within this group are further identified based on a predetermined percentage of those in the threshold group such as, for example, those in the top 25%, the top 20%, the top 15%, the top 10%, the top 5%, or the top 1%. The particular percentage chosen is dependent on the strength of the association of the target nucleic acid sequence with the target-specific oligonucleotide probe, on the relative concentration of the target nucleotide sequence and the interfering sequence, and the like. The percentages may be related to the particular scoring scheme used. For example, where the scoring scheme involves predicted melting temperatures (Tm), those oligonucleotide probes having a Tm that is within 10 degrees, or within 5 degrees, of that of a perfect match would be under consideration for potentially addressing a sequence that would result in substantial interference with detection of a target nucleic acid sequence. Where the scoring scheme involves predicted free energy of interaction, those oligonucleotide probes having a ΔG that is within 3 kcal/mole, or within 1.5 kcal/mole, of that of a perfect match would be under consideration for potentially addressing a sequence that would result in substantial interference with detection of a target nucleic acid sequence.

Setting the threshold involves two factors. The factor relates to how many positions on an array are available or, stated another way, how many probes can one afford to synthesize for the array. The second factor relates to experimental experience. Candidate cross-hybridization probes are subjected to experimental analysis to determine how well a particular scoring scheme is working to identify cross-hybridization probes to interfering sequences that are of most concern with respect to a particular target polynucleotide. Part of this latter factor relates to how many cross-hybridization oligonucleotide probes are necessary to address such interfering sequences.

A cross-hybridization event may be evaluated using any method that will allow such an evaluation. Examples of such methods, by way of illustration and not limitation, are discussed in detail below. Cross-hybridization levels are estimated employing the target-specific hybridization oligonucleotide probe and a related cross-hybridization oligonucleotide probe. Based on the cross-hybridization results, the target-specific hybridization oligonucleotide probe is included in, or excluded from, the set of target-specific hybridization oligonucleotide probes used to perform a particular assay. The above steps may be repeated to determine a set of target-specific hybridization oligonucleotide probes and a set of cross-hybridization oligonucleotide probes, both of the sets comprising one or a minimum number of such probes.

A primary focus of the present invention is to provide for more efficient design of target-specific hybridization oligonucleotide probes and/or cross-hybridization oligonucleotide probes and thereby to reduce to a minimum the number of such probes that are utilized in analyzing a target nucleotide sequence. In one aspect the present approach is directed to the design of controls in nucleic acid hybridization assays. Once the controls are designed, the resulting selected cross-hybridization probes may be used in experiments with actual samples, which may be limited in amount. Accordingly, the amount of actual test sample employed in an analysis may be conserved.

The present invention addresses a potential situation that is of concern. In a complex sample, there may be one or more target sequences, i.e., interfering sequences, which form an imperfect match with a particular oligonucleotide probe, which then hybridizes with such sequence as well as to the target nucleic sequence if present. The potential for interfering sequences is increased where the length of the hybridization oligonucleotide is relatively long, i.e., longer than about 30 nucleotides. The point is, however, that binding to this oligonucleotide probe produces a signal whether or not the target nucleotide sequence is present. This signal is interpreted as detection of the intended target, leading to a false-positive assay result.

In the present invention a set of cross-hybridization oligonucleotide probes is selected for each target nucleotide sequence. The set of probes comprises a minimum number, which is less that a full set, of cross-hybridization oligonucleotide probes for each target nucleotide sequence. The selection of this minimal set is performed by taking advantage of knowledge of other, related sequences present in the target sample, constructing cross-hybridization probes or probe mixtures that effectively model multiple mismatched target possibilities, or combining these two approaches. The cross-hybridization results obtained with the set either target cross-hybridization events having a predetermined probability, e.g., the most likely or most probable cross-hybridization events, or are substantially the same as an average of results obtained with a larger number, such as a full set, of cross-hybridization oligonucleotide probes. In one aspect of the present method, the set of target-specific and matched cross-hybridization oligonucleotide probes is contacted with a target nucleotide sequence. The differential hybridization of the target-specific and matched cross-hybridization oligonucleotide probes to the target sample is determined, and the specificity of hybridization of the target-specific probe to its intended target sequence is estimated employing the cross-hybridization results.

Cross-hybridization oligonucleotide probes are oligonucleotide probes that may be used in conjunction with the target-specific hybridization oligonucleotide probes. The cross-hybridization (or mismatch) oligonucleotide probes are directed to sequences (interfering sequences or inappropriate sequences) that may be present that are capable of hybridizing with the target-specific oligonucleotide probe. If the target-specific hybridization oligonucleotide probe is 25 bases long, the number of potential cross-hybridizing sequences is about 4²⁵ or 1.13×10¹⁵. If the target-specific hybridization oligonucleotide probe is 60 bases long, the number of potential cross-hybridizing sequences is 4⁶⁰ or 1.33×10³⁶. Many of these possibilities yield duplex free energies so unfavorable that they need not be considered. However, even if one restricts oneself to consideration of the number of 60-mer sequences that differ in 4 positions from a given 60-mer probe, the number of potential cross-hybridizing sequences is 3⁴×60×59×58×57=9.5×10⁸. Obviously, a real experiment can sample only a tiny fraction of these possibilities; even experiments that employ a seeming abundance of cross-hybridization probes are, in reality, measuring results from a sparse collection of the possible cross-hybridization events.

As mentioned above, a minimum number of cross-hybridization oligonucleotide probes are utilized in the present invention for each target nucleic acid sequence. The minimum number is less than a larger number, such as a full set, of cross-hybridization oligonucleotide probes for each target nucleic acid sequence. The minimum number of cross-hybridization oligonucleotide probes is dependent on the nature of the target nucleic acid sequence and the nature and number of sequences that may interfere with the detection of the target nucleic acid sequence as explained more fully below. Usually, the minimum number of cross-hybridization oligonucleotide probes per gene is no more than about 10, more usually, no more than about 5, and may be as few as one. This is to be contrasted with a larger number of cross-hybridization oligonucleotide probes that are used in the prior art. The number of such probes per gene in prior art methods of gene expression-level measurement is usually at least about 20 and may be as high as 100 or more.

The focus of the present invention is to use as few a number of cross-hybridization oligonucleotide probes as necessary to achieve the level of specificity and sensitivity achieved with a larger number of such probes. Desirably, a single cross-hybridization oligonucleotide probe is determined and used. On the other hand, a set of cross-hybridization oligonucleotide probes may be determined wherein the cross-hybridization result obtained with the set measures the extent of occurrence of hybridization events that have a predetermined probability based on certain information about the target nucleic acid sequence and the target-specific oligonucleotide probe. Accordingly, the cross-hybridization results obtained with the minimum number of cross-hybridization probes indicate that cross-hybridization is or is not a problem as well as or better than the results obtained with a larger number of such probes. The results are obtained from an analysis, or hybridization study, of a sample suspected of containing a target nucleotide sequence using a target-specific hybridization oligonucleotide probe and one or more cross-hybridization oligonucleotide probes. The results are usually determined by measuring the signal produced in the analysis after hybridization studies have been conducted. Specificity ratios (i.e. the ratio of net signal from the target-specific hybridization oligonucleotide probe to the average of the signals from the matched cross-hybridization probes) greater than 2 are suggestive of a target-specific hybridization oligonucleotide probe of requisite specificity. Specificity ratios greater than 5 are generally interpreted as indicators of a target-specific hybridization oligonucleotide probe having good specificity.

As mentioned above, cross-hybridization probes of the present invention are produced by certain specified deletions from hybridization oligonucleotides selected as described above. The cross-hybridization probes of the invention are unambiguous for any given position in the probe, since there is only one way to delete a base. The effects on binding of probe produced by deletions are approximately equivalent to the effects of a single base substitution at the same position; therefore, the signal from a deletion probe can be interpreted by methods similar to those used to interpret mismatch control probe signals. It is also noteworthy that any apparatus that can synthesize polynucleotide arrays can synthesize the cross-hybridization probes of the invention without modification of the apparatus.

In the present methods, a hybridization experiment is carried out using a candidate hybridization oligonucleotide probe that is specific for a particular target nucleic acid sequence, which is selected in accordance with the methods discussed above. The intensity of signal is measured. Based on the level of signal, the candidate hybridization oligonucleotide probe may be chosen for further experimentation or redesigned using an approach such as that described above. Cross-hybridization oligonucleotide probes are then selected based on the candidate probe according to the above described method.

An experiment is then conducted using the hybridization oligonucleotide probe and the cross-hybridization probe. The sample containing target nucleic acid sequence and interfering sequence is placed on the surface of a support, to which the sequences bind. Then, the surface is contacted with the above oligonucleotide probes, which are labeled, and signal is measured. If the intensity of the signal from the target-specific probe is at a level that is considered reasonable, i.e., sufficiently detectable, and the intensity of signal from the cross-hybridization probe is negligible, then the interfering sequence is not much cause for concern. However, if the intensity of the signal from the cross-hybridization probe is equal to or greater than 10% of the signal from the target-specific probe, the interfering sequence may present a problem in an assay for the target nucleic acid sequence. The cross-hybridization signal should not exceed 75% of the Perfect match probe signal. In such a circumstance, additional evaluations or experiments in accordance with the present invention may be carried out to examine other cross-hybridization probes. In this manner the design of probes is perfected to achieve a set comprising a minimum number of cross-hybridization probes that provide the appropriate level of sensitivity and specificity. When this set of cross-hybridization oligonucleotide probes is employed with samples of unknown content, one has a higher degree of confidence that the results obtained are reliable.

The additional evaluations that may be carried out include searching for a different target-specific oligonucleotide probe that does not exhibit a potential for cross-hybridization and verifying that cross-hybridization is taking place by experimentally observing it. Picking a different probe is by far the easiest approach, if satisfactory alternative candidates are available. If the particulars of the experiment dictate that even a probe of mediocre specificity cannot be rejected, then the actual specificity of the probe can be measured by producing a synthetic version of the polynucleotide corresponding to the sequence to which the cross-hybridization probe hybridizes, using means well known to the art. This sequence or “cross-hybridization target” is then labeled in a manner easily distinguished from the normal experimental sample (e.g., a different, spectrally distinct fluorophore). The probe array is contacted with a mixture of the natural, complex sample and the synthetic sample, and the result of the contacting is determined. The result is usually determined by examining the array for the presence of hybrids. In this case signals from hybrids involving the target-specific probes and the cross-hybridization probes are observed. If the cross-hybridization target shows significant binding to the original target-specific probe, then the probe is not specific and should not be used without using the results of the cross-hybridization target experiment to correct for cross-hybridization. If the cross-hybridization target shows low binding to the original target-specific oligonucleotide probe and significant binding to the cross-hybridization probe, then the original cross-hybridization result is explained and can be dismissed. Significant binding is defined as at least 10% of the signal observed from the original target-specific probe with the original target. If neither probe shows significant binding of the cross-hybridization target, then the original result is unexplained, and there may be a problem with cross-hybridization to a third, unidentified target.

As mentioned above, one aspect of the present invention is a method for analyzing a target nucleic acid sequence. A set of target-specific oligonucleotide probes for the target nucleic acid sequence is selected. The method may involve one or more iterations of a process that comprises identifying a cross-hybridization oligonucleotide probe based on a candidate target-specific oligonucleotide probe for the target nucleic acid sequence, determining cross-hybridization results employing the cross-hybridization oligonucleotide probe and target-specific oligonucleotide probe together with a sample containing the target nucleic acid sequence and an interfering nucleic acid sequence, and including or excluding the target-specific oligonucleotide probe in the set based on the cross-hybridization results. The cross-hybridization oligonucleotide probe measures the extent of the occurrence of a hybridization event of a predetermined probability between the target-specific oligonucleotide probe and an interfering sequence, which may be present in the sample containing the target nucleic acid sequence. The process is repeated until a set of target-specific oligonucleotide probes is identified.

In the method of analysis the set of target-specific oligonucleotide probes is contacted with a sample suspected of containing a target nucleic acid sequence, and the extent of hybridization of the target-specific oligonucleotide probes to the target nucleic acid sequence is determined. During the analysis the sample may be contacted with one or more of the cross-hybridization oligonucleotide probes identified above. The use of such cross-hybridization probes would depend on whether sample-to-sample variation is such that cross-hybridization of the target-specific oligonucleotide probe and an interfering nucleic acid sequence may be a problem. In other words, although the present method may be used to select a set of target-specific oligonucleotide probes of high specificity, some samples to be tested may contain more of an interfering nucleic acid sequence than other samples. Alternatively, the best set of target-specific oligonucleotide probes obtained may still have some cross-hybridization with interfering nucleic acid sequences even though the amount of such interfering sequences does not vary significantly from one sample to the next. The method of the present invention provides an added advantage in that one may correct for cross-hybridization problems using the cross-hybridization probes identified by the present methods. By employing cross-hybridization oligonucleotide probes in accordance with the present invention, the relative amount of an interfering sequence can be measured and the overall signal obtained may be corrected to reflect only the amount of the target nucleic acid sequence.

The cross-hybridization oligonucleotide probe used in the above analysis may be a single probe obtained as described above. On the other hand the cross-hybridization probe may be part of a set of oligonucleotide probes wherein the cross-hybridization result obtained with the set is representative of a cross-hybridization event of a predetermined probability between the target-specific oligonucleotide probe and an interfering nucleic acid sequence.

As mentioned above, the methods and reagents of the present invention are particularly useful in the area of oligonucleotide arrays. One aspect of the present invention is an addressable array comprising a support having a surface, a spot on the surface having bound thereto an oligonucleotide probe specific for a target nucleic acid sequence and at least one spot on the surface having bound thereto a cross-hybridization oligonucleotide probe wherein the cross-hybridization oligonucleotide probe measures the extent of the occurrence of a cross-hybridization event of a predetermined probability between an interfering nucleic acid sequence and the oligonucleotide probe specific for a target nucleic acid sequence. The probes are employed in an effective amount, namely, an amount that will yield the desired result such as detection of the target nucleic acid sequence.

A method for detecting a target nucleic acid sequence comprises contacting a medium suspected of containing the target nucleic acid sequence with the above addressable array and determining a result of the contacting. The result indicates the presence or absence of the target nucleic acid sequence in the medium. The result may be determined by examining the array for the presence of a hybrid of the target nucleic acid sequence and the oligonucleotide probe specific for the target nucleic acid sequence. The presence of the hybrid indicates the presence of the target nucleic acid sequence in the medium. In one approach the target nucleic acid sequence is labeled and the result is determined by examining the array for the presence of signal associated with the label, the signal being related to the presence of the hybrid. One aspect of the invention is the product of the above method, namely, the assay result, which may be evaluated at the site of the testing or it may be shipped to a remote location, e.g., another site, for evaluation and communication to an interested party.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

As mentioned above, the methods of the present invention are preferably carried out at least in part with the aid of a computer. The considerations regarding the computer, computer software, and the like are similar or the same as those discussed above. A computer program may be utilized to carry out the above method steps including identifying suitable cross-hybridization probes. The computer program provides for (i) input of hybridization oligonucleotide sequence information, (ii) efficient algorithms for computation of cross-hybridization oligonucleotide probes, (iii) efficient, versatile mechanisms for filtering sets of oligonucleotide sequences based on parameter values, (iv) mechanisms for measurement of cross-hybridization results employing cross-hybridization oligonucleotide probes and hybridization oligonucleotide probes, and (v) mechanisms for outputting the results to provide for selecting or rejecting a particular hybridization oligonucleotide probe for the set of such probes in accordance with the method of the present invention in a versatile, machine-readable or human-readable form. As mentioned above, the output may be directed to a manufacturing apparatus for synthesizing oligonucleotides.

Another aspect of the present invention is a computer program product comprising a computer readable storage medium having a computer program stored thereon which, when loaded into a computer, selects a set of target-specific oligonucleotide probes for use in analyzing a target nucleic acid sequence. The computer program performs steps comprising (a) identifying under computer control a cross-hybridization oligonucleotide probe based on hybridization oligonucleotide identified as described above wherein the cross-hybridization oligonucleotide probe measures the extent of the occurrence of a cross-hybridization event having a predetermined probability, (b) determining under computer control cross-hybridization results employing the cross-hybridization oligonucleotide probe and the hybridization oligonucleotide probe and (c) selecting or rejecting under computer control the hybridization oligonucleotide probe for the set based on the cross-hybridization results.

As indicated above, any of the steps of the methods of the present invention can be executed on a suitable computer system. The computer system may be programmed from a computer readable storage medium that carries code for the system to execute the steps required of it. The computer readable storage medium may comprise, for example, magnetic storage media such as optical disc, optical tape, or machine readable bar code, solid state electronic storage devices such as random access memory (RAM), or read only memory (ROM), or any other physical device or medium that might be employed to store a computer program. It will also be understood that computer systems of the present invention can include the foregoing programmable systems and/or hardware or hardware/software combinations that can execute the same or equivalent steps.

The computer-based method may be carried out by using the following exemplary computer system. Input means is provided for introducing a hybridization oligonucleotide sequence into the computer system. The input means may permit manual input of the hybridization oligonucleotide sequence. The input means may also be a database or a standard format file such as GenBank. Also included is means for determining a cross-hybridization oligonucleotide probe based on the hybridization oligonucleotide sequence wherein the cross-hybridization oligonucleotide probe measures the extent of the occurrence of a cross-hybridization event having a predetermined probability. Suitable means is a computer program or software, which also provides memory means for determining and storing cross-hybridization results employing the cross-hybridization oligonucleotide probe and hybridization oligonucleotide probe. The computer system further comprises means for controlling the computer system to select or reject the hybridization oligonucleotide probe for the set based on the cross-hybridization results. Suitable means is a computer program or software such as, for example, Microsoft® Excel spreadsheet, Microsoft® Access relational database or the like, which also provides memory means for storing selection results. The computer system also comprises means for outputting data relating to the selection results. Such means may be machine readable or human readable and may be software that communicates with a printer, electronic mail, another computer program, and the like.

### Other Embodiments

A method for predicting the potential of a hybridization oligonucleotide of length greater than about 20 nucleotides to hybridize to a target nucleotide sequence, the method comprising:

(a) identifying a predetermined number of unique oligonucleotides of at least about 20 nucleotides in length within a nucleotide sequence of at least about 30 nucleotides in length that is hybridizable with the target nucleotide sequence, the oligonucleotides being chosen to sample the entire length of the nucleotide sequence,

(b) determining and evaluating for each of the oligonucleotides at least one parameter that is independently predictive of the ability of each of the oligonucleotides to hybridize to the target nucleotide sequence,

(c) selecting a subset of oligonucleotides within the predetermined number of unique oligonucleotides based on an examination of the parameter and application of a rule that rejects some of the oligonucleotides of step (b),

(d) identifying oligonucleotides in the selected subset, viewed according to order of position along the nucleotide sequence, that are in clusters along a region of the nucleotide sequence and that identify a contig in the nucleotide sequence,

(e) selecting for each cluster a hybridization oligonucleotide that comprises at least a portion of the nucleotide sequence of the contig and, if not the entire nucleotide sequence of the contig, an additional nucleotide sequence adjacent the contig. In one embodiment the hybridization oligonucleotide comprises at least a portion of the nucleotide sequence of said contig wherein said hybridization oligonucleotide is longer in length than any of the oligonucleotides in (d) which identify the contig. The hybridization oligonucleotide may comprise at least a portion of the nucleotide sequence of the contig, usually, at least the entire sequence of the contig or a portion of the sequence of the contig plus a sequence of nucleotides, which is not in the contig and which corresponds to a sequence of nucleotides of the nucleotide sequence where such sequence of nucleotides is adjacent the contig. In one embodiment the hybridization oligonucleotide comprises at least the entire nucleotide sequence of the contig. In another embodiment the hybridization oligonucleotide comprises at least one half of the nucleotide sequence including a central nucleotide of the contig plus a sequence of nucleotides that is not in the contig and that corresponds to a sequence of nucleotides of the nucleotide sequence where such sequence of nucleotides is adjacent the contig.

A method according to the above wherein in step (e) a hybridization oligonucleotide is selected for each cluster, in descending order of cluster rank, wherein the hybridization oligonucleotide has as its central nucleotide the central nucleotide of a region of the nucleotide sequence that corresponds to the cluster, the remaining nucleotides of the hybridization oligonucleotide being added, in correspondence to the nucleotides in the nucleotide sequence that extend in one or both directions from the central nucleotide, until a predetermined length of the hybridization oligonucleotide is obtained and a predetermined number of hybridization oligonucleotides are obtained, one from each cluster, wherein the higher the rank of the cluster, the higher the hybridization potential of the hybridization oligonucleotide for the target nucleotide sequence.

A method for predicting the potential of a hybridization oligonucleotide of length greater than about 20 nucleotides to hybridize to a target nucleotide sequence, said method comprising:

(a) identifying a predetermined number of unique oligonucleotides of at least about 20 nucleotides in length within a nucleotide sequence of at least about 30 nucleotides in length that is hybridizable with said target nucleotide sequence, said oligonucleotides being chosen to sample the entire length of said nucleotide sequence,

(b) determining and evaluating for each of said oligonucleotides at least one parameter that is independently predictive of the ability of each of said oligonucleotides to hybridize to said target nucleotide sequence,

(c) selecting a subset of oligonucleotides within said predetermined number of unique oligonucleotides based on an examination of said parameter and application of a rule that rejects some of said oligonucleotides of step (b),

(d) identifying oligonucleotides in said selected subset, viewed according to order of position along said nucleotide sequence, that are in clusters along a region of said nucleotide sequence,

(e) ranking said clusters in order of number of oligonucleotides in each cluster from greatest number to least number of oligonucleotides,

(f) selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence.

A method according to the above wherein the length of a hybridization oligonucleotide is equal to the length of said region of said nucleotide sequence.

A method according to the above wherein the length of a hybridization oligonucleotide is greater than the length of said region of said nucleotide sequence.

A method according to the above wherein said unique oligonucleotides are of identical length N.

A method according to the above wherein said unique oligonucleotides are spaced one nucleotide apart, said predetermined number comprising L−N+1 oligonucleotides.

A method according to the above wherein said parameter is selected from the group consisting of composition factors, thermodynamic factors, chemosynthetic efficiencies, kinetic factors and empirical factors.

A method according to the above wherein said parameter is a composition factor selected from the group consisting of mole fraction (G+C), percent (G+C), sequence complexity, and sequence information content.

A method according to the above wherein said parameter is a thermodynamic factor selected from the group consisting of predicted duplex melting temperature, predicted enthalpy of duplex formation, predicted entropy of duplex formation, predicted free energy of duplex formation, predicted melting temperature of the most stable intramolecular structure of the oligonucleotide or its complement, predicted enthalpy of the most stable intramolecular structure of the oligonucleotide or its complement, predicted entropy of the most stable intramolecular structure of the oligonucleotide or its complement, predicted free energy of the most stable intramolecular structure of the oligonucleotide or its complement, predicted melting temperature of the most stable hairpin structure of the oligonucleotide or its complement, predicted enthalpy of the most stable hairpin structure of the oligonucleotide or its complement, predicted entropy of the most stable hairpin structure of the oligonucleotide or its complement, predicted free energy of the most stable hairpin structure of the oligonucleotide or its complement, thermodynamic partition function for intramolecular structure of the oligonucleotide or its complement.

A method according to the above wherein said parameter is derived from a factor by mathematical transformation of said factor.

A method according to the above wherein said parameter is a chemosynthetic efficiency selected from the group consisting of coupling efficiencies and overall efficiency of the synthesis of a target nucleotide sequence or an oligonucleotide probe.

A method according to the above wherein said parameter is a kinetic factor selected from the group consisting of steric factors calculated via molecular modeling, rate constants calculated via molecular dynamics simulations, rate constants calculated via semi-empirical kinetic modeling, associative rate constants, dissociative rate constants, enthalpies of activation, entropies of activation, and free energies of activation.

A method according to the above wherein said parameters are determined for said oligonucleotides by means of a computer program.

A method according to the above wherein said hybridization oligonucleotides are subsequently attached to a surface.

A method according to the above wherein said hybridization oligonucleotides are DNA.

A method according to the above wherein said hybridization oligonucleotides are RNA.

A method according to the above wherein said hybridization oligonucleotides contain chemically modified nucleotides.

A method according to the above wherein said target nucleotide sequence is RNA.

A method according to the above wherein said target nucleotide sequence is DNA.

A method according to the above wherein said target nucleotide sequence contains chemically modified nucleotides.

A method according to the above wherein said parameter is, for each oligonucleotide:target nucleotide sequence duplex, the difference between the predicted duplex melting temperature corrected for salt concentration and the temperature of hybridization of each of said hybridization oligonucleotides with said target nucleotide sequence.

A method according to the above wherein step (c) comprises identifying a subset of oligonucleotides within said predetermined number of unique oligonucleotides by establishing cut-off values for said parameter.

A method according to the above wherein said step (c) comprises identifying a subset of oligonucleotides within said predetermined number of unique oligonucleotides by converting the values of said parameter into a dimensionless number.

A method according to the above wherein step (b) comprises determining at least two parameters wherein said parameters are poorly correlated with respect to one another.

A method according to the above wherein said parameters are derived from a combination of factors by mathematical transformation of those factors.

A method according to the above, which comprises (i) identifying a subset of oligonucleotides within said predetermined number of unique oligonucleotides by establishing cut-off values for each of said parameters.

A method according to the above, which comprises determining the sizes of, said clusters of step (d) by counting the number of contiguous oligonucleotides in said region of said hybridizable sequence.

A method according to the above which comprises determining the sizes of said clusters of step (d) by counting the number of oligonucleotides in said subset that begin in a region of predetermined length in said hybridizable sequence.

A method according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained

A method according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained.

A method for predicting the potential of a hybridization oligonucleotide of length greater than about 20 nucleotides to hybridize to a target nucleotide sequence, said method comprising:

(a) identifying a set of overlapping oligonucleotides of at least about 20 nucleotides in length within a nucleotide sequence of at least about 30 nucleotides in length that is complementary to said target nucleotide sequence,

(b) determining and evaluating for each of said oligonucleotides at least two parameters that are independently predictive of the ability of each of said oligonucleotides to hybridize to said target nucleotide sequence wherein said parameters are poorly correlated with respect to one another,

(c) selecting a subset of oligonucleotides within said predetermined number of oligonucleotides based on an examination of said parameter and application of a rule that rejects some of said oligonucleotides of step (b),

(d) identifying oligonucleotides in said selected subset, viewed according to order of position along said nucleotide sequence, that are in clusters along a region of said nucleotide sequence,

(e) ranking said clusters in order of number of oligonucleotides in each cluster from greatest number to least number of oligonucleotides,

(e) selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence.

A method according to the above wherein the length of a hybridization oligonucleotide is equal to the length of said region of said nucleotide sequence.

A method according to the above wherein the length of a hybridization oligonucleotide is greater than the length of said region of said nucleotide sequence.

A method according to the above wherein said oligonucleotides are of identical length N.

A method according to the above wherein said oligonucleotides are spaced one nucleotide apart, said predetermined number comprising L−N+1 oligonucleotides.

A method according to the above wherein said parameter is selected from the group consisting of composition factors, thermodynamic factors, chemosynthetic efficiencies, kinetic factors and empirical factors.

A method according to the above wherein said parameter is a composition factor selected from the group consisting of mole fraction (G+C), percent (G+C), sequence complexity, and sequence information content.

A method according to the above wherein said parameter is a thermodynamic factor selected from the group consisting of predicted duplex melting temperature, predicted enthalpy of duplex formation, predicted entropy of duplex formation, predicted free energy of duplex formation, predicted melting temperature of the most stable intramolecular structure of the oligonucleotide or its complement, predicted enthalpy of the most stable intramolecular structure of the oligonucleotide or its complement, predicted entropy of the most stable intramolecular structure of the oligonucleotide or its complement, predicted free energy of the most stable intramolecular structure of the oligonucleotide or its complement, predicted melting temperature of the most stable hairpin structure of the oligonucleotide or its complement, predicted enthalpy of the most stable hairpin structure of the oligonucleotide or its complement, predicted entropy of the most stable hairpin structure of the oligonucleotide or its complement, predicted free energy of the most stable hairpin structure of the oligonucleotide or its complement, thermodynamic partition function for intramolecular structure of the oligonucleotide or its complement.

A method according to the above wherein said parameter is derived from a factor by mathematical transformation of said factor.

A method according to the above wherein said parameter is a chemosynthetic efficiency selected from the group consisting of coupling efficiencies and overall efficiency of the synthesis of a target nucleotide sequence or an oligonucleotide probe.

A method according to the above wherein said parameter is a kinetic factor selected from the group consisting of steric factors calculated via molecular modeling, rate constants calculated via molecular dynamics simulations, rate constants calculated via semi-empirical kinetic modeling, associative rate constants, dissociative rate constants, enthalpies of activation, entropies of activation, and free energies of activation.

A method according to the above wherein said parameters are determined for said oligonucleotides by means of a computer program.

A method according to the above wherein said hybridization oligonucleotides are subsequently attached to a surface.

A method according to the above wherein said hybridization oligonucleotides are DNA.

A method according to the above wherein said hybridization oligonucleotides are RNA.

A method according to the above wherein said hybridization oligonucleotides contain chemically modified nucleotides.

A method according to the above wherein said target nucleotide sequence is RNA.

A method according to the above wherein said target nucleotide sequence is DNA.

A method according to the above wherein said target nucleotide sequence contains chemically modified nucleotides.

A method according to the above wherein said parameter is, for each oligonucleotide:target nucleotide sequence duplex, the difference between the predicted duplex melting temperature corrected for salt concentration and the temperature of hybridization of each of said hybridization oligonucleotides with said target nucleotide sequence.

A method according to the above wherein step (c) comprises identifying a subset of oligonucleotides within said predetermined number of unique oligonucleotides by establishing cut-off values for said parameter.

A method according to the above wherein said step (c) comprises identifying a subset of oligonucleotides within said predetermined number of unique oligonucleotides by converting the values of said parameter into a dimensionless number.

A method according to the above wherein step (b) comprises determining at least two parameters wherein said parameters are poorly correlated with respect to one another.

A method according to the above wherein said parameters are derived from a combination of factors by mathematical transformation of those factors.

A method according to the above, which comprises (i) identifying a subset of oligonucleotides within said predetermined number of unique oligonucleotides by establishing cut-off values for each of said parameters.

A method according to the above, which comprises determining the sizes of, said clusters of step (d) by counting the number of contiguous oligonucleotides in said region of said hybridizable sequence.

A method according to the above which comprises determining the sizes of said clusters of step (d) by counting the number of oligonucleotides in said subset that begin in a region of predetermined length in said hybridizable sequence.

A method according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained

A method according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained

A method for predicting the potential of a hybridization oligonucleotide of at least about 20 nucleotides in length to hybridize to a complementary target nucleotide sequence, said method comprising:

(a) obtaining, from a nucleotide sequence at least about 30 nucleotides in length and complementary to said target nucleotide sequence, a set of overlapping oligonucleotides of at least about 20 nucleotides in length and of identical length N and spaced one nucleotide apart, said set comprising L−N+1 oligonucleotides,

(b) determining and evaluating for each of said oligonucleotides the parameters: (i) the predicted melt temperature of the duplex of said oligonucleotide and said target nucleotide sequence corrected for salt concentration and (ii) predicted free energy of the most stable intramolecular structure of the oligonucleotide at the temperature of hybridization of each of said oligonucleotides with said target nucleotide sequence,

(c) selecting a subset of oligonucleotides within said predetermined number of oligonucleotides based on an examination of said parameter and application of a rule that rejects some of said oligonucleotides of step (b),

(d) identifying oligonucleotides in said selected subset, viewed according to order of position along said nucleotide sequence, that are in clusters along a region of said nucleotide sequence,

(e) ranking said clusters in order of number of oligonucleotides in each cluster from greatest number to least number of oligonucleotides,

(e) selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence.

A method according to the above wherein the length of a hybridization oligonucleotide is equal to the length of said region of said nucleotide sequence.

A method according to the above wherein the length of a hybridization oligonucleotide is greater than the length of said region of said nucleotide sequence.

A method according to the above wherein said parameters are derived by mathematical transformation of the factors named in step (b).

A method according to the above wherein the melting temperature of step (b) is transformed by subtracting the temperature of hybridization.

A method according to the above wherein said parameters are determined for said oligonucleotides by means of a computer program.

A method according to the above wherein said oligonucleotides are attached to a surface.

A method according to the above wherein said oligonucleotides are DNA or RNA.

A method according to the above wherein said oligonucleotides contain chemically modified nucleotides.

A method according to the above wherein said target nucleotide sequence is RNA or DNA.

A method according to the above wherein said target nucleotide sequence contains chemically modified nucleotides.

A method according to the above where clustering is determined by calculating a moving window-averaged combination score <Sᵢ> for the ith probe by the equation:

$$⟨ {S}_{i} ⟩ = \frac{1}{w} {\sum }_{j = i - \frac{w - 1}{2}}^{i + \frac{w - 1}{2}} {S}_{j} ,$$

w=an odd integer,

where w is the length of the window for averaging, and then applying a cutoff filter to the value of <Sᵢ>.

A method according to the above wherein said hybridization oligonucleotide is at least 30 nucleotides in length.

A method according to the above wherein said hybridization oligonucleotide is at least 50 nucleotides in length.

A method according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained

A method according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained

A computer based method for predicting the potential of a hybridization oligonucleotide of length greater than about 20 nucleotides to hybridize to a target nucleotide sequence, said method comprising:

(a) identifying under computer control a predetermined number of unique oligonucleotides of at least about 20 nucleotides in length within a nucleotide sequence of at least about 30 nucleotides in length that is hybridizable with said target nucleotide sequence, said oligonucleotides being chosen to sample the entire length of said nucleotide sequence,

(b) under computer control determining and evaluating for each of said oligonucleotides at least one parameter that is independently predictive of the ability of each of said oligonucleotides to hybridize to said target nucleotide sequence,

(c) selecting under computer control a subset of oligonucleotides within said predetermined number of unique oligonucleotides based on an examination of said parameter and application of a rule that rejects some of said oligonucleotides of step (b),

(d) identifying under computer control oligonucleotides in said selected subset, viewed according to order of position along said nucleotide sequence, that are in clusters along a region of said nucleotide sequence,

(e) ranking under computer control said clusters in order of number of oligonucleotides in each cluster from greatest number to least number of oligonucleotides,

(e) under computer control selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence.

A computer based method according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained.

A computer based method according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained

A computer system for conducting a method for predicting the potential of a hybridization oligonucleotide to hybridize to a target nucleotide sequence, said system comprising:

(a) input means for introducing a target nucleotide sequence into said computer system,

(b) means for determining a number of unique oligonucleotide sequences that are within a nucleotide sequence that is hybridizable with said target nucleotide sequence, said oligonucleotide sequences being chosen to sample the entire length of said nucleotide sequence,

(c) memory means for storing said oligonucleotide sequences,

(d) means for controlling said computer system to carry out a determination and evaluation for each of said oligonucleotide sequences a value for at least one parameter that is independently predictive of the ability of each of said oligonucleotide sequences to hybridize to said target nucleotide sequence,

(e) means for storing said parameter values,

(f) means for controlling said computer to carry out an identification from said stored parameter values a subset of oligonucleotide sequences within said number of unique oligonucleotide sequences based on said evaluation of said parameter,

(g) means for storing said subset of oligonucleotides,

(h) means for controlling said computer to carry out an identification of oligonucleotide sequences in said subset that are clustered along a region of said nucleotide sequence that is hybridizable to said target nucleotide sequence.

(i) means for storing said oligonucleotide sequences in said subset,

(j) means for outputting data relating to said oligonucleotide sequences in said subset,

(k) means for ranking said clusters in order of number of oligonucleotides in each cluster from greatest number to least number of oligonucleotides,

(l) means for selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence, and

(m) means for outputting data relating to said hybridization oligonucleotides.

A computer system according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained

A computer system according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained

A method for selecting a cross-hybridization oligonucleotide probe for use in conjunction with a hybridization oligonucleotide of at least about 30 nucleotides in length for analyzing a target nucleotide sequence, said method comprising:

(a) identifying a hybridization oligonucleotide of at least 30 nucleotides in length that is specific for said target nucleotide sequence,

(b) selecting a cross-hybridization oligonucleotide probe based on said hybridization oligonucleotide of step (a) by a process comprising deletion of multiple nucleotides from said hybridization oligonucleotide.

A method for selecting a cross-hybridization oligonucleotide probe in length for use in conjunction with a hybridization oligonucleotide of at least about 20 nucleotides for analyzing a target nucleotide sequence, said method comprising:

(a) identifying a hybridization oligonucleotide by a method according to the above,

(b) selecting a cross-hybridization oligonucleotide probe based on said hybridization oligonucleotide by a process comprising deletion of at least one nucleotide from said hybridization oligonucleotide wherein said deletion(s) are evenly spaced with respect to the nucleotides of said hybridization oligonucleotide and the number of deletions is based on the length of said cross-hybridization oligonucleotide probe.

A method according to the above wherein said hybridization oligonucleotide is at least about 50 nucleotides in length and said deletions of step (b) are at a frequency of about one for every 20 to 25 nucleotides of said hybridization oligonucleotide.

A method according to the above wherein said hybridization oligonucleotide is 60 nucleotides in length and said deletions are made at nucleotides 15, 30 and 45 of said hybridization oligonucleotide.

A method for selecting a cross-hybridization oligonucleotide probe in length for use in conjunction with a hybridization oligonucleotide of at least about 20 nucleotides for analyzing a target nucleotide sequence, said method comprising:

(a) identifying a hybridization oligonucleotide of at least 20 nucleotides in length that is specific for said target nucleotide sequence,

(b) selecting a cross-hybridization oligonucleotide probe based on said hybridization oligonucleotide of step (a) by a process comprising deletion of at least one nucleotide from said hybridization oligonucleotide wherein said deletion(s) are evenly spaced with respect to the nucleotides of said hybridization oligonucleotide.

A method according to the above wherein said hybridization oligonucleotide is at least about 50 nucleotides in length and said deletions of step (b) are at a frequency of one for about every 20 to 25 nucleotides of said hybridization oligonucleotide.

A method according to the above wherein said hybridization oligonucleotide is 60 nucleotides in length and said deletions are made at nucleotides 15, 30 and 45 of said hybridization oligonucleotide.

A method for selecting a hybridization oligonucleotide of at least about 20 nucleotides in length for hybridization to a target nucleotide sequence and for selecting a cross-hybridization probe corresponding to said hybridization oligonucleotide, said method comprising:

(a) identifying a predetermined number of unique oligonucleotides of at least about 20 nucleotides in length within a nucleotide sequence of at least about 30 nucleotides in length that is hybridizable with said target nucleotide sequence, said oligonucleotides being chosen to sample the entire length of said nucleotide sequence,

(b) determining and evaluating for each of said oligonucleotides at least one parameter that is independently predictive of the ability of each of said oligonucleotides to hybridize to said target nucleotide sequence,

(c) selecting a subset of oligonucleotides within said predetermined number of unique oligonucleotides based on an examination of said parameter and application of a rule that rejects some of said oligonucleotides of step (b),

(d) identifying oligonucleotides in said selected subset, viewed according to order of position along said nucleotide sequence, that are in clusters along a region of said nucleotide sequence,

(e) ranking said clusters in order of number of oligonucleotides in each cluster from greatest number to least number of oligonucleotides,

(e) selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence, and

(f) selecting a cross-hybridization oligonucleotide probe based on said hybridization oligonucleotide of step (e) by a process comprising deletion of at least one nucleotide from said hybridization oligonucleotide wherein said deletion(s) are evenly spaced with respect to the nucleotides of said hybridization oligonucleotide and the number of deletions is based on the length of said cross-hybridization oligonucleotide probe.

A method according to the above wherein the length of a hybridization oligonucleotide is equal to the length of said region of said nucleotide sequence.

A method according to the above wherein the length of a hybridization oligonucleotide is greater than the length of said region of said nucleotide sequence.

A method according to the above wherein said unique oligonucleotides are of identical length N.

A method according to the above wherein said unique oligonucleotides are spaced one nucleotide apart, said predetermined number comprising L−N+1 oligonucleotides.

A method according to the above wherein said parameter is selected from the group consisting of composition factors, thermodynamic factors, chemosynthetic efficiencies and kinetic factors.

A method according to the above wherein said parameter is a composition factor selected from the group consisting of mole fraction (G+C), percent (G+C), sequence complexity, and sequence information content.

A method according to the above wherein said parameter is a thermodynamic factor selected from the group consisting of predicted duplex melting temperature, predicted enthalpy of duplex formation, predicted entropy of duplex formation, predicted free energy of duplex formation, predicted melting temperature of the most stable intramolecular structure of the oligonucleotide or its complement, predicted enthalpy of the most stable intramolecular structure of the oligonucleotide or its complement, predicted entropy of the most stable intramolecular structure of the oligonucleotide or its complement, predicted free energy of the most stable intramolecular structure of the oligonucleotide or its complement, predicted melting temperature of the most stable hairpin structure of the oligonucleotide or its complement, predicted enthalpy of the most stable hairpin structure of the oligonucleotide or its complement, predicted entropy of the most stable hairpin structure of the oligonucleotide or its complement, predicted free energy of the most stable hairpin structure of the oligonucleotide or its complement, thermodynamic partition function for intramolecular structure of the oligonucleotide or its complement.

A method according to the above wherein said parameters are determined for said oligonucleotides by means of a computer program.

A method according to the above wherein said hybridization oligonucleotides are subsequently attached to a surface.

A method according to the above wherein said hybridization oligonucleotides are DNA or RNA.

A method according to the above wherein said target nucleotide sequence is RNA or DNA.

A method according to the above wherein step (b) comprises determining at least two parameters wherein said parameters are poorly correlated with respect to one another.

A method according to the above wherein said hybridization oligonucleotide is at least about 50 nucleotides in length and said deletions of step (b) are at a frequency of about one for every 20 to 25 nucleotides of said hybridization oligonucleotide.

A method according to the above wherein said hybridization oligonucleotide is 60 nucleotides in length and said deletions are made at nucleotides 15, 30 and 45 of said hybridization oligonucleotide.

A method according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained

A method according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained

A computer based method for selecting a hybridization oligonucleotide of at least about 20 nucleotides in length for hybridization to a target nucleotide sequence and for selecting a cross-hybridization probe corresponding to said hybridization oligonucleotide, said method comprising:

(a) identifying under computer control a predetermined number of unique oligonucleotides of at least about 20 nucleotides in length within a nucleotide sequence of at least about 30 nucleotides in length that is hybridizable with said target nucleotide sequence, said oligonucleotides being chosen to sample the entire length of said nucleotide sequence,

(b) under computer control determining and evaluating for each of said oligonucleotides at least one parameter that is independently predictive of the ability of each of said oligonucleotides to hybridize to said target nucleotide sequence,

(c) selecting under computer control a subset of oligonucleotides within said predetermined number of unique oligonucleotides based on an examination of said parameter and application of a rule that rejects some of said oligonucleotides of step (b),

(d) identifying under computer control oligonucleotides in said selected subset, viewed according to order of position along said nucleotide sequence, that are in clusters along a region of said nucleotide sequence,

(e) ranking under computer control said clusters in order of number of oligonucleotides in each cluster from greatest number to least number of oligonucleotides,

(e) selecting under computer control for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence, and

(f) selecting under computer control a cross-hybridization oligonucleotide probe based on said hybridization oligonucleotide of step (e) by a process comprising deletion of at least one nucleotide from said hybridization oligonucleotide wherein said deletion(s) are evenly spaced with respect to the nucleotides of said hybridization oligonucleotide.

A method according to the above wherein the remaining nucleotides of said hybridization oligonucleotide are added, in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained

A method according to the above wherein the remaining nucleotides of said hybridization oligonucleotide are added, in correspondence to the nucleotides in said nucleotide sequence that extend in only one direction from said central nucleotide, until a predetermined length of said hybridization oligonucleotide is obtained and a predetermined number of hybridization oligonucleotides are obtained

A method for selecting a set of target-specific oligonucleotide probes of at least about 20 nucleotides in length for use in analyzing a target nucleotide sequence, said method comprising:

(a) identifying a cross-hybridization oligonucleotide probe based on said target nucleic acid sequence by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe,

(b) determining cross-hybridization results employing said cross-hybridization oligonucleotide probe and target-specific oligonucleotide probe and

(c) selecting or rejecting said target-specific oligonucleotide probe for said set based on said cross-hybridization results.

A method according to the above wherein said cross-hybridization oligonucleotide probe is at least about 50 nucleotides in length and said deletions of step (a) are at a frequency of one for about every 20 to 25 nucleotides of said hybridization oligonucleotide.

A method according to the above wherein said cross-hybridization oligonucleotide probe is 60 nucleotides in length and said deletions are made at nucleotides 15, 30 and 45 of said hybridization oligonucleotide.

A method according to the above wherein said cross-hybridization oligonucleotide probe is 60 nucleotides in length and said deletions are made at nucleotides 12, 24, 36 and 48 of said hybridization oligonucleotide.

A computer based method for selecting a set of target-specific oligonucleotide probes of at least about 20 nucleotides in length for use in analyzing a target nucleotide sequence, said method comprising:

(a) identifying under computer control a cross-hybridization oligonucleotide probe based on said target nucleic acid sequence by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe,

(b) determining under computer control cross-hybridization results employing said cross-hybridization oligonucleotide probe and target-specific oligonucleotide probe and

(c) under computer control selecting or rejecting said target-specific oligonucleotide probe for said set based on said cross-hybridization results.

A method for detecting differences between an individual sequence and a known reference sequence, said method comprising:

(a) combining under hybridization conditions labeled individual sequence, a surface bound reference oligonucleotide probe based on said known reference sequence and a set of surface bound deletion oligonucleotide probes of at least about 20 nucleotides in length wherein said set of deletion oligonucleotide probes is prepared by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe,

(b) determining hybridization ratios for said set of deletion oligonucleotide probes with respect to said reference oligonucleotide probe and

(c) relating said hybridization ratios to the presence or absence of differences between said individual sequence and said reference sequence.

A method according to the above wherein said deletion oligonucleotide probe is at least about 50 nucleotides in length and said deletions are at a frequency of one for about every 20 to 25 nucleotides of said deletion oligonucleotide probe.

A method according to the above wherein said deletion oligonucleotide probe is 60 nucleotides in length and said deletions are made at nucleotides 15, 30 and 45 of said deletion oligonucleotide probe.

A method according to the above wherein said differences are mutations.

A method according to the above wherein said differences are single nucleotide polymorphisms.

An addressable array comprising:

(a) a support having a surface,

(b) a spot on said surface having bound thereto an oligonucleotide probe specific for a target nucleic acid sequence and

(c) at least one spot on said surface having bound thereto a cross-hybridization oligonucleotide probe wherein said cross-hybridization oligonucleotide probe measures the extent of the occurrence of a cross-hybridization event of a predetermined probability between an interfering nucleic acid sequence and said oligonucleotide probe specific for a target nucleic acid sequence wherein said cross-hybridization oligonucleotide probe is selected by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe.

An array according to the above wherein said cross-hybridization oligonucleotide probe is at least about 50 nucleotides in length and said deletions are at a frequency of one for about every 20 to 25 nucleotides of said cross-hybridization oligonucleotide probe.

An array according to the above wherein said cross-hybridization oligonucleotide probe is 60 nucleotides in length and said deletions are made at nucleotides 15, 30 and 45 of said cross-hybridization oligonucleotide probe.

An array according to the above wherein said cross-hybridization oligonucleotide probe is 60 nucleotides in length and said deletions are made at nucleotides 12, 24, 36 and 48 of said cross-hybridization oligonucleotide probe.

A method for detecting a target nucleic acid sequence, said method comprising:

(a) contacting a medium suspected of containing said target nucleic acid sequence with the above array and

(b) determining a result of said contacting, said result indicating the presence or absence of said target nucleic acid sequence in said medium.

A method according to the above wherein said determining of said result comprises examining said array for the presence of a hybrid of said target nucleic acid sequence and said oligonucleotide probe specific for said target nucleic acid sequence, the presence thereof indicating the presence of said target nucleic acid sequence in said medium.

A method according to the above wherein said target nucleic acid sequence is labeled and said result is determined by examining said array for the presence of signal associated with said label, said signal being related to the presence of said hybrid.

An assay result determined by a method according to the above.

A method comprising forwarding to a remote location a result according to the above.

An addressable array comprising:

(a) a support having a surface,

(b) a spot on said surface having bound thereto an oligonucleotide probe specific for a target nucleic acid sequence and

(c) at least one spot on said surface having bound thereto a cross-hybridization oligonucleotide probe wherein said cross-hybridization oligonucleotide probe measures the extent of the occurrence of a cross-hybridization event of a predetermined probability between an interfering nucleic acid sequence and said oligonucleotide probe specific for a target nucleic acid sequence wherein said cross-hybridization oligonucleotide probe is selected by a method according to Claim 1.

An array according to the above wherein said cross-hybridization oligonucleotide probe is at least about 50 nucleotides in length and said deletions are at a frequency of one for about every 20 to 25 nucleotides of said cross-hybridization oligonucleotide probe.

An array according to the above wherein said cross-hybridization oligonucleotide probe is 60 nucleotides in length and said deletions are made at nucleotides 15, 30 and 45 of said cross-hybridization oligonucleotide probe.

An array according to the above wherein said cross-hybridization oligonucleotide probe is 60 nucleotides in length and said deletions are made at nucleotides 12, 24, 36 and 48 of said cross-hybridization oligonucleotide probe.

A method for detecting a target nucleic acid sequence, said method comprising:

(a) contacting a medium suspected of containing said target nucleic acid sequence with the above array and

(b) determining a result of said contacting, said result indicating the presence or absence of said target nucleic acid sequence in said medium.

A method according to the above said determining of said result comprises examining said array for the presence of a hybrid of said target nucleic acid sequence and said oligonucleotide probe specific for said target nucleic acid sequence, the presence thereof indicating the presence of said target nucleic acid sequence in said medium.

A method according to the above wherein said target nucleic acid sequence is labeled and said result is determined by examining said array for the presence of signal associated with said label, said signal being related to the presence of said hybrid.

An assay result determined by a method according to the above.

A method comprising forwarding to a remote location a result according to the above.

A method for predicting the potential of a hybridization oligonucleotide of at least about 20 nucleotides in length to hybridize to a complementary target nucleotide sequence, said method comprising:

(a) obtaining, from a nucleotide sequence at least about 30 nucleotides in length L and complementary to said target nucleotide sequence, a set of overlapping oligonucleotides of at least about 20 nucleotides in length and of identical length N and spaced S nucleotides apart, said set comprising 1+Int[(L−N)/S] oligonucleotides, wherein “Int” is the integer part of the indicated quotient,

(b) determining experimentally and evaluating for each of said oligonucleotides the hybridization of said oligonucleotides with said target nucleotide sequence,

(c) selecting a subset of oligonucleotides within said predetermined number of oligonucleotides based on said evaluation and application of a rule that rejects some of said oligonucleotides of step (b),

(d) identifying oligonucleotides in said selected subset, viewed according to order of position along said nucleotide sequence, that are in clusters along a region of said nucleotide sequence,

(e) ranking said clusters in order of number of oligonucleotides in each cluster from greatest number to least number of oligonucleotides,

(f) selecting for each cluster, in descending order of cluster rank, a hybridization oligonucleotide that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence.

A method according to the above wherein step (b) comprises synthesizing said oligonucleotides of step (a) and experimentally testing the hybridization of said oligonucleotides with an array comprising said target nucleotide sequence.

Kits of the Invention

Another aspect of the present invention relates to kits useful for conveniently performing a method in accordance with the invention. To enhance the versatility of the subject invention, the reagents can be provided in packaged combination, in the same or separate containers, so that the ratio of the reagents provides for substantial optimization of the method. The reagents may each be in separate containers or various reagents can be combined in one or more containers depending on the cross-reactivity and stability of the reagents.

In one embodiment a kit comprises an oligonucleotide probe that is specific for the target nucleic acid sequence and a cross-hybridization oligonucleotide probe based on a candidate hybridization oligonucleotide probe for the target nucleic acid sequence. The hybridization oligonucleotide probes may comprise a label. The cross-hybridization oligonucleotide probe measures the occurrence of a cross-hybridization event of predetermined probability between an interfering nucleic acid sequence and the hybridization oligonucleotide probe specific for the target nucleic acid sequence. In one aspect the cross-hybridization results obtained with the cross-hybridization oligonucleotide probe, which may be a single probe or a set comprising a minimum number of such probes, are substantially the same as an average of results obtained with the full set of cross-hybridization oligonucleotide probes.

The kit can further include other separately packaged reagents for conducting the method as well as ancillary reagents and so forth. The relative amounts of the various reagents in the kits can be varied widely to provide for concentrations of the reagents that substantially optimize the reactions that need to occur during the present method. Under appropriate circumstances one or more of the reagents in the kit can be provided as a dry powder, usually lyophilized, including excipients, which on dissolution will provide for a reagent solution having the appropriate concentrations for performing a method in accordance with the present invention. The kit can further include a written description of a method in accordance with the present invention as described above.

The reagents, methods and kits of the invention are useful for, among others, mutation detection, mutation identification, polymorphism analysis, genotyping, de novo sequencing, re-sequencing, gene expression profiling, cDNA clustering and the like.

It should be understood that the above description is intended to illustrate and not limit the scope of the invention. Other aspects, advantages and modifications within the scope of the invention will be apparent to those skilled in the art to which the invention pertains. The invention has application to biopolymers in general such as, for example, polynucleotides, poly (amino acids), e.g., proteins and peptides, and the like. Factors in the application of the present invention to a particular biopolymer include the ability of the biopolymer to show homology phenomena that can be studied and the availability of a reasonable method for scoring such homology phenomena. In application of the present invention to biopolymers in general the term “hybridizing” used herein would have the more general meaning of “binding” between biopolymers. The following examples are put forth so as to provide those of ordinary skill in the art with examples of how to make and use the method and products of the invention, and are not intended to limit the scope of what the inventors regard as their invention.

### EXAMPLES

The invention is demonstrated further by the following illustrative examples. Parts and percentages are by weight unless otherwise indicated. Temperatures are in degrees Centigrade (° C.) unless otherwise specified. The following preparations and examples illustrate the invention but are not intended to limit its scope. All reagents used herein were from Amresco, Inc., Solon, Ohio (buffers), Pharmacia Biotech, Piscataway, N.J. (nucleoside triphosphates) or Promega, Madison, Wis. (RNA polymerases) unless indicated otherwise.

### Example 1

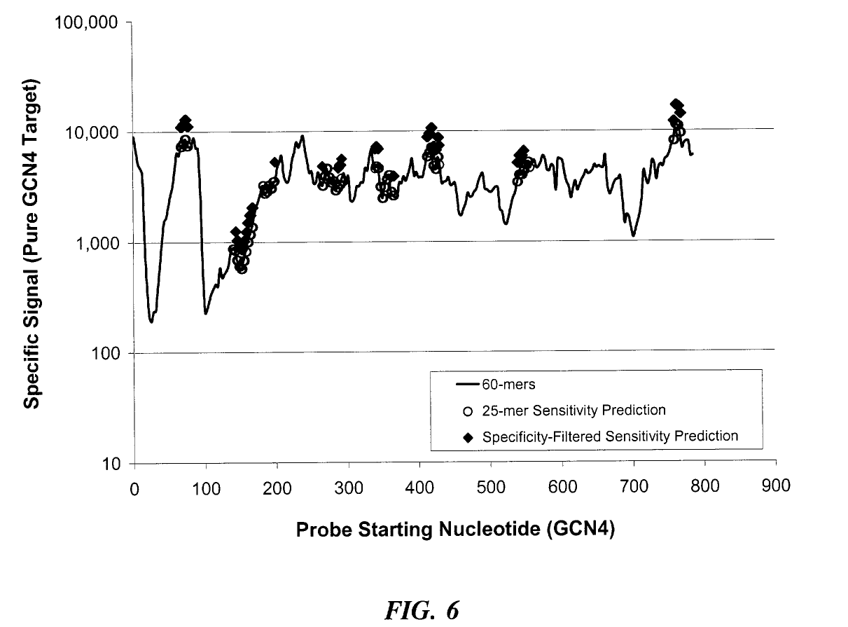

The method of the present invention was tested for its ability to identify sensitive and specific 60-mer probes to the yeast gene GCN4 (Systematic Name YEL009C). The coding sequence of GCN4 was obtained from the National Center for Biotechnology Information, via their Entrez web service (http://www.ncbi.nlm.nih.gov/). Predictor 25-mer probes were designed by the method of Shannon, et al., U.S. Pat. No. 6,251,588, the relevant portions of which are incorporated herein by reference. All possible 25-mer probes were examined (i.e., spacing of 1 nucleotide between possible probes). The hybridization predictor parameters employed were duplex Tₘ and probe self-structure free energy (ΔG_{M}). Range filters were employed for both parameters. The Tₘ filter was 64° C.≦Tₘ≦69° C.; the ΔG_{M} filter was 0.0 kcal/mole≦ΔG_{M}≦20 kcal/mole. The probes that passed both filters were scored for their ability to form contiguous sets of probes (“contigs”); contigs of length 10 or less were discarded. The results of this process constituted the sensitivity prediction. The comparison of the sensitivity prediction to the results of an experiment in which pure GCN4 target was hybridized to an array bearing a tiling pattern of 60-mer probes (3 nucleotide spacing) to GCN4 is shown in FIG. 6. The experimental conditions and array design employed were the same as those reported by Hughes, et al. (2001), “Expression profiling using microarrays fabricated by an ink-jet oligonucleotide synthesizer”, Nat Biotechnol 19(4): 342-7, except that 4.92 M urea was substituted for formamide as a hybridization modifier. FIG. 6 shows that the predictions of the method of Shannon, et al. successfully identified regions with high hybridization potential, and that extension of the predicted regions by the method of the present invention results in the picking of sensitive 60-mer probes.

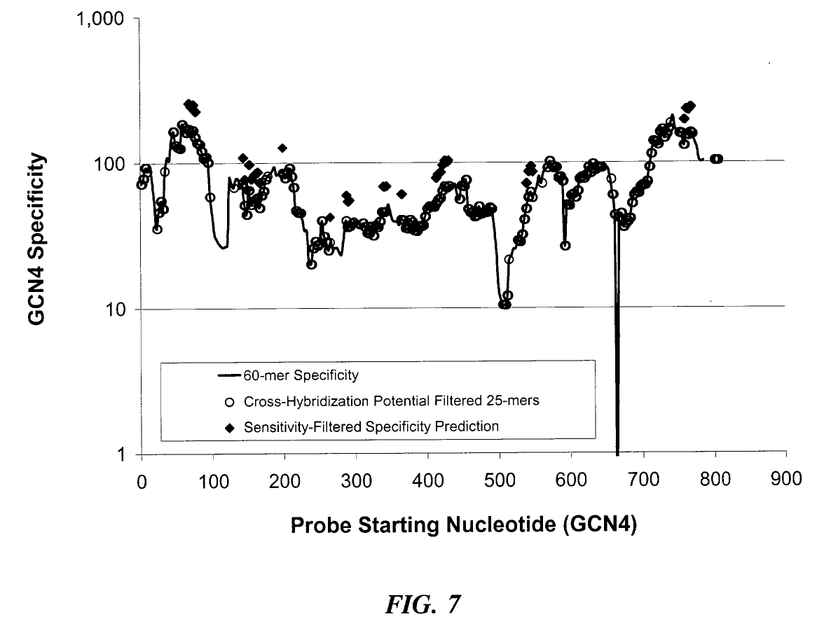

The specificity of predictor 25-mers was estimated by homology searching. Briefly, the program BLAST (Altschul et al (1990), “Basic local alignment search tool”, J Mol Biol 215(3): 403-10) was used to search a database containing all predicted open reading frames (ORF's) from yeast. Matches with a BLAST E-value between 0.0 and 0.3 were counted, and probes with a total count exceeding 1 were discarded. The results of this prediction are shown in FIG. 7 (open circles) superimposed on the experimentally measured specificity for the system. Specificity was measured by the method of Hughes, et al., with the modifications described above. Briefly, a labeled sample from a yeast gcn4/gcn4 knockout strain was hybridized to the array used to measure sensitivity. The label employed a different color channel than that used by the pure GCN4 target. Specificity was calculated as the ratio of the specific signal to the cross-hybridization (i.e., knockout channel) signal. It is clear from FIG. 7 that the specificity predictions cluster (form contigs) in the regions that yielded the highest experimental specificities.

Combined sensitivity and specificity predictions were performed by intersecting the results of the sensitivity prediction and the results of the specificity prediction. These probes are shown as filled diamonds in FIG. 6 and FIG. 7. It is clear from the figures that the combined predictions, after extension by the method of the present invention, yield 60-mer probes that are both sensitive and specific.

### Example 2

The method of the current invention was put into practice in a study designed to determine the performance of 60-mer probes extended from 25-mer probes. Eight 25-mer probes were designed to each of 50 genes of Saccharomyces cerevisiae by the method of Shannon, et al., U.S. Pat. No. 6,251,588. Briefly, all possible 25-mer probes over the 3′ 1000 base pairs (bp) of each gene were interrogated. The hybridization predictor parameters employed were duplex predicted melting temperature (64° C.≦Tm≦69° C.) and probe self-structure free energy (0.0 kcal/mole≦ΔGm≦20 kcal/mol). Probes were then interrogated by homology searching against all other yeast predicted genes using BLAST (Altschul, et al. (1990), “Basic local alignment search tool”, J Mol Biol 215(3):403-10) to search a database containing all open reading frames (ORF's) of yeast. Matches with a BLAST E-value between 0.0 and 0.3 were counted and probes with a total count exceeding 1 were discarded. Finally, probes that passed all three filters were scored for their ability to form contiguous sets of probes (contigs); contigs of 3 or fewer probes were discarded. Following all of the above filtering, the central probes from the 8 longest contigs were selected for each of the 50 genes included in the analysis. Matching 60-mer probes were then designed from these probes by extending the sequence from the 3′ end in accordance with the present invention.

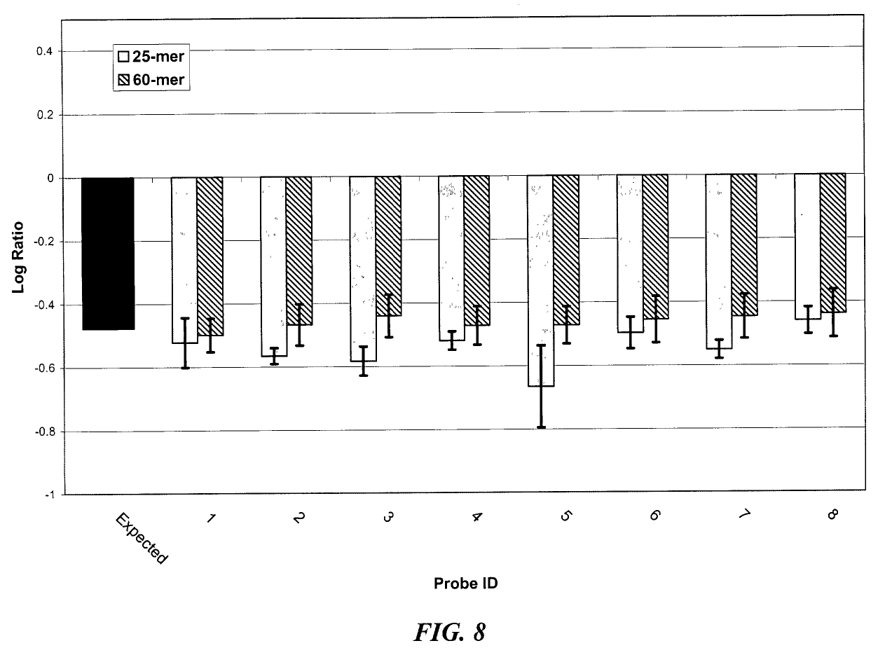

Microarrays were designed containing both the 25-mer and 60-mer probes for these 50 selected genes. The microarrays were hybridized according to the manufacturer's instructions (Agilent Technologies, Palo Alto, Calif.) with a complex target consisting of yeast cRNA generated from a knockout yeast strain (yel009c/yel009c) and labeled synthetic transcript representing the knocked out gene (YEL009c) at a defined ratio. As expected, the 60-mer probes showed approximately 10-fold higher intensity than the comparable 25-mer probe for the genes analyzed. Specificity was measured as the accuracy of detecting the spike-in log ratio. Accuracy of log ratio results for the spike-in experiment is shown in FIG. 8. The results demonstrate that the 60-mers designed from the 25-mers in accordance with the present invention had comparable accuracy of log ratio to the 25-mers and therefore had no loss of specificity.

### Example 3

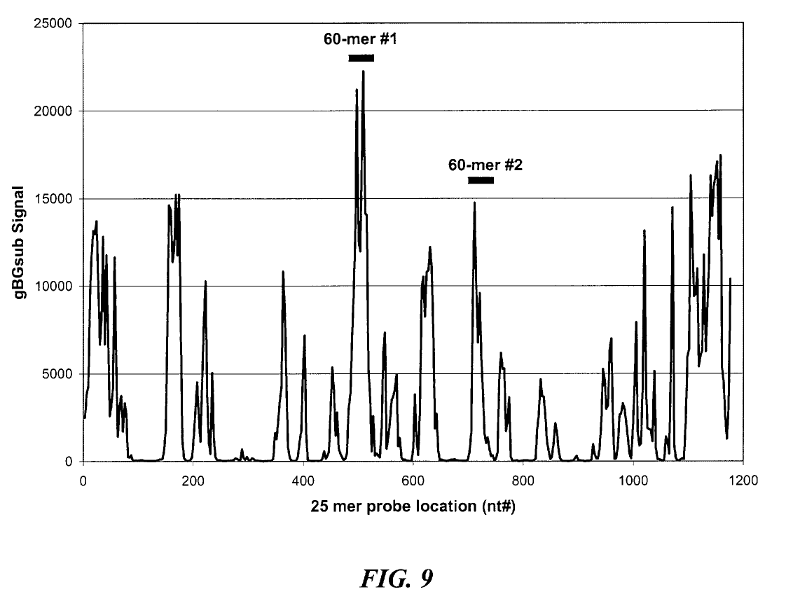

The method of the current invention was put into practice using empirically tested 25-mer probes to design 60-mer probes. An array was designed to contain 25-mers spanning the sequences of 4 different genes from Arabadopisis and E. coli. The probes were designed by generating 25-mer probes from the sequences of these genes, spacing by 3 (i.e., probes starting at bases 3, 6, 9,12, etc.). Arrays generated with this design were hybridized individually with synthetic labeled transcripts representing each of the 4 genes. Analysis of probe performance was based on the signal intensity as a function of probe position. From this analysis, regions of the gene where 25-mer probes gave high signals were identified, and 60-mers were designed to be contained within this region. If no region of greater than 60 bases could be identified, 60-mer probes were designed to be centered on the longest region of high signal intensity. The results as depicted in FIG. 9 demonstrate the intensity as a function of probe position for one of the genes and illustrate the regions chosen for 60-mer probes.

All publications and patent applications cited in this specification are herein incorporated by reference as if each individual publication or patent application where specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for predicting the potential of a hybridization oligonucleotide of length greater than about 20 nucleotides to hybridize to a target nucleotide sequence, said method comprising:
(a) identifying a predetermined number of unique oligonucleotides of at least about 20 nucleotides in length within a nucleotide sequence of at least about 30 nucleotides in length that is hybridizable with said target nucleotide sequence, said oligonucleotides being chosen to sample a length of said nucleotide sequence,
(b) determining and evaluating for said oligonucleotides at least one parameter that is predictive of the ability of said oligonucleotides to hybridize to said target nucleotide sequence,
(c) selecting a subset of oligonucleotides within said predetermined number of unique oligonucleotides based on an examination of said parameter and application of a rule that rejects some of said oligonucleotides of step (b),
(d) identifying oligonucleotides in said selected subset, viewed according to order of position along said nucleotide sequence, that are in clusters along a region of said nucleotide sequence and that identify a contig in said nucleotide sequence,
(e) selecting for a cluster a hybridization oligonucleotide that comprises at least a portion of the nucleotide sequence of said contig wherein said hybridization oligonucleotide is different from any of the oligonucleotides in (d) which identify the contig.

2. A method according to claim 1 wherein in step (e) a hybridization oligonucleotide is selected for each cluster, in descending order of cluster rank, wherein said hybridization oligonucleotide has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until a predetermined length of said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence.

3. A method according to claim 1 wherein the hybridization oligonucleotide in (e) has a sequence overlapping multiple oligonucleotides in (d) which identify the contig.

4. A method according to claim 1 wherein said hybridization oligonucleotide is longer than any of the oligonucleotides in (d).

5. A method for predicting the potential of a hybridization oligonucleotide of length greater than about 20 nucleotides to hybridize to a target nucleotide sequence, said method comprising:
(a) identifying a predetermined number of unique oligonucleotides of at least about 20 nucleotides in length within a nucleotide sequence of at least about 30 nucleotides in length that is hybridizable with said target nucleotide sequence, said oligonucleotides being chosen to sample the entire length of said nucleotide sequence,
(b) determining and evaluating for each of said oligonucleotides at least one parameter that is independently predictive of the ability of each of said oligonucleotides to hybridize to said target nucleotide sequence,
(c) selecting a subset of oligonucleotides within said predetermined number of unique oligonucleotides based on an examination of said parameter and application of a rule that rejects some of said oligonucleotides of step (b),
(d) identifying oligonucleotides in said selected subset, viewed according to order of position along said nucleotide sequence, that are in clusters along a region of said nucleotide sequence,
(e) ranking said clusters in order of number of oligonucleotides in each cluster from greatest number to least number of oligonucleotides,
(f) selecting for each cluster, in descending order of cluster rank, a hybridization oligonucleotide of predetermined length that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence and wherein said hybridization oligonucleotide is different from any of said oligonucleotides in said cluster.

6. A method according to claim 5 wherein the length of a hybridization oligonucleotide is equal to, or greater than, the length of said region of said nucleotide sequence.

7. A method according to claim 5 wherein said unique oligonucleotides are of identical length N and wherein said unique oligonucleotides are spaced one nucleotide apart, said predetermined number comprising L−N+1 oligonucleotides.

8. A method according to claim 5 wherein said parameter is selected from the group consisting of composition factors, thermodynamic factors, chemosynthetic efficiencies, kinetic factors and empirical factors.

9. A method according to claim 5 wherein said hybridization oligonucleotides are subsequently attached to a surface.

10. A method according to claim 5 wherein said target nucleotide sequence is DNA or RNA.

11. A method according to claim 5 wherein step (c) comprises identifying a subset of oligonucleotides within said predetermined number of unique oligonucleotides by establishing cut-off values for said parameter.

12. A method according to claim 5, which comprises determining the sizes of said clusters of step (d) by counting the number of contiguous oligonucleotides in said region of said hybridizable sequence.

13. A method according to claim 5 which comprises determining the sizes of said clusters of step (d) by counting the number of oligonucleotides in said subset that begin in a region of predetermined length in said hybridizable sequence.

14. A method according to claim 5 wherein the remaining nucleotides of said hybridization oligonucleotide are added (i) in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained or (ii) are added, in correspondence to the nucleotides in said nucleotide sequence that extend in only one direction from said central nucleotide, until a predetermined length of said hybridization oligonucleotide is obtained and a predetermined number of hybridization oligonucleotides are obtained.

15. A method according to claim 5 wherein step (b) comprises determining and evaluating for each of said oligonucleotides at least two parameters that are independently predictive of the ability of each of said oligonucleotides to hybridize to said target nucleotide sequence wherein said parameters are poorly correlated with respect to one another.

16. A method for predicting the potential of a hybridization oligonucleotide of at least about 20 nucleotides in length to hybridize to a complementary target nucleotide sequence, said method comprising:
(a) obtaining, from a nucleotide sequence at least about 30 nucleotides in length and complementary to said target nucleotide sequence, a set of overlapping oligonucleotides of at least about 20 nucleotides in length and of identical length N and spaced one nucleotide apart, said set comprising L−N+1 oligonucleotides,
(b) determining and evaluating for each of said oligonucleotides the parameters: (i) the predicted melt temperature of the duplex of said oligonucleotide and said target nucleotide sequence corrected for salt concentration and (ii) predicted free energy of the most stable intramolecular structure of the oligonucleotide at the temperature of hybridization of each of said oligonucleotides with said target nucleotide sequence,
(c) selecting a subset of oligonucleotides within said predetermined number of oligonucleotides based on an examination of said parameter and application of a rule that rejects some of said oligonucleotides of step (b),
(d) identifying oligonucleotides in said selected subset, viewed according to order of position along said nucleotide sequence, that are in clusters along a region of said nucleotide sequence,
(e) ranking said clusters in order of number of oligonucleotides in each cluster from greatest number to least number of oligonucleotides,
(f) selecting for each cluster, in descending order of cluster rank, a hybridization oligonucleotide of predetermined length that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence and wherein said predetermined length of said hybridization oligonucleotide is greater than the length of any of said oligonucleotides in said cluster.

17. A method according to claim 16 wherein the length of a hybridization oligonucleotide is equal to, or greater than, the length of said region of said nucleotide sequence.

18. A method according to claim 16 wherein said oligonucleotides are attached to a surface.

19. A method according to claim 16 wherein said hybridization oligonucleotide is at least 30 nucleotides in length.

20. A method according to claim 16 wherein the remaining nucleotides of said hybridization oligonucleotide are added (i) in correspondence to the nucleotides in said nucleotide sequence that extend equally or unequally in both directions from said central nucleotide, until a predetermined length of said hybridization oligonucleotide is obtained and a predetermined number of hybridization oligonucleotides are obtained or (ii) are added, in correspondence to the nucleotides in said nucleotide sequence that extend in only one direction from said central nucleotide, until a predetermined length of said hybridization oligonucleotide is obtained and a predetermined number of hybridization oligonucleotides are obtained.

21. A method according to claim 16, which is a computer based method wherein steps (a) through (f) are carried Out under computer control.

22. A computer system for conducting a method for predicting the potential of a hybridization oligonucleotide to hybridize to a target nucleotide sequence, said system comprising:
(a) input means for introducing a target nucleotide sequence into said computer system,
(b) means for determining a number of unique oligonucleotide sequences that are within a nucleotide sequence that is hybridizable with said target nucleotide sequence, said oligonucleotide sequences being chosen to sample the entire length of said nucleotide sequence,
(c) memory means for storing said oligonucleotide sequences,
(e) means for controlling said computer system to carry out a determination and evaluation for each of said oligonucleotide sequences a value for at least one parameter that is independently predictive of the ability of each of said oligonucleotide sequences to hybridize to said target nucleotide sequence,
(e) means for storing said parameter values,
(f) means for controlling said computer to carry out an identification from said stored parameter values a subset of oligonucleotide sequences within said number of unique oligonucleotide sequences based on said evaluation of said parameter,
(g) means for storing said subset of oligonucleotides,
(h) means for controlling said computer to carry out an identification of oligonucleotide sequences in said subset that are clustered along a region of said nucleotide sequence that is hybridizable to said target nucleotide sequence.
(i) means for storing said oligonucleotide sequences in said subset,
(j) means for outputting data relating to said oligonucleotide sequences in said subset,
(k) means for ranking said clusters in order of number of oligonucleotides in each cluster from greatest number to least number of oligonucleotides,
(l) means for selecting for each cluster, in descending order of cluster rank, a hybridization oligonucleotide of predetermined length that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence and wherein said predetermined length of said hybridization oligonucleotide is greater than the length of any of said oligonucleotides in said cluster, and
(m) means for outputting data relating to said hybridization oligonucleotides.

23. A method for selecting a cross-hybridization oligonucleotide probe for use in conjunction with a hybridization oligonucleotide of at least about 20 nucleotides for analyzing a target nucleotide sequence, said method comprising:
(a) identifying a hybridization oligonucleotide by a method according to claim 5,
(b) selecting a cross-hybridization oligonucleotide probe based on said hybridization oligonucleotide by a process comprising deletion of at least one nucleotide from said hybridization oligonucleotide wherein said deletion(s) are evenly spaced with respect to the nucleotides of said hybridization oligonucleotide and the number of deletions is based on the length of said cross-hybridization oligonucleotide probe.

24. A method according to claim 23 wherein said hybridization oligonucleotide is at least about 50 nucleotides in length and said deletions of step (b) are at a frequency of about one for every 20 to 25 nucleotides of said hybridization oligonucleotide.

25. A method according to claim 23 wherein said hybridization oligonucleotide is 60 nucleotides in length and said deletions are made at nucleotides 15, 30 and 45 of said hybridization oligonucleotide.

26. A method for selecting a cross-hybridization oligonucleotide probe for use in conjunction with a hybridization oligonucleotide of at least about 20 nucleotides in length for analyzing a target nucleotide sequence, said method comprising:
(a) identifying a hybridization oligonucleotide of at least 20 nucleotides in length that is specific for said target nucleotide sequence,
(b) selecting a cross-hybridization oligonucleotide probe based on said hybridization oligonucleotide of step (a) by a process comprising deletion of at least one nucleotide from said hybridization oligonucleotide wherein said deletion(s) are evenly spaced with respect to the nucleotides of said hybridization oligonucleotide.

27. A method according to claim 26 wherein said hybridization oligonucleotide is at least about 50 nucleotides in length and said deletions of step (b) are at a frequency of one for about every 20 to 25 nucleotides of said hybridization oligonucleotide.

28. A method according to claim 26 wherein said hybridization oligonucleotide is 60 nucleotides in length and said deletions are made at nucleotides 15, 30 and 45 of said hybridization oligonucleotide.

29. A method according to claim 26 wherein said hybridization 30 oligonucleotide of at least about 20 nucleotides in length is selected by a method comprising:
(a) identifying a predetermined number of unique oligonucleotides of at least about 20 nucleotides in length within a nucleotide sequence of at least about 30 nucleotides in length that is hybridizable with said target nucleotide sequence, said oligonucleotides being chosen to sample the entire length of said nucleotide sequence,
(b) determining and evaluating for each of said oligonucleotides at least one parameter that is independently predictive of the ability of each of said oligonucleotides to hybridize to said target nucleotide sequence,
(c) selecting a subset of oligonucleotides within said predetermined number of unique oligonucleotides based on an examination of said parameter and application of a rule that rejects some of said oligonucleotides of step (b),
(d) identifying oligonucleotides in said selected subset, viewed according to order of position along said nucleotide sequence, that are in clusters along a region of said nucleotide sequence,
(e) ranking said clusters in order of number of oligonucleotides in each cluster from greatest number to least number of oligonucleotides, and
(f) selecting for each cluster, in descending order of cluster rank, a hybridization oligonucleotide of predetermined length that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence and wherein said predetermined length of said hybridization oligonucleotide is greater than the length of any of said oligonucleotides in said cluster.

30. A method according to claim 29 wherein said parameter is selected from the group consisting of composition factors, thermodynamic factors, chemosynthetic efficiencies and kinetic factors.

31. A method according to claim 29 wherein said hybridization oligonucleotides are subsequently attached to a surface.

32. A method according to claim 29, which is a computer based method wherein each of said steps is carried out under computer control.

33. A method for selecting a set of target-specific oligonucleotide probes of at least about 20 nucleotides in length for use in analyzing a target nucleotide sequence, said method comprising:
(a) identifying a cross-hybridization oligonucleotide probe based on said target nucleic acid sequence by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe,
(b) determining cross-hybridization results employing said cross-hybridization oligonucleotide probe and target-specific oligonucleotide probe and
(c) selecting or rejecting said target-specific oligonucleotide probe for said set based on said cross-hybridization results.

34. A method according to claim 33 wherein said cross-hybridization oligonucleotide probe is at least about 50 nucleotides in length and said deletions of step (a) are at a frequency of one for about every 20 to 25 nucleotides of said hybridization oligonucleotide.

35. A method according to claim 33, which is a computer based method wherein steps (a)-(c) are carried out under computer control.

36. A method for detecting differences between an individual sequence and a known reference sequence, said method comprising:
(a) combining under hybridization conditions labeled individual sequence, a surface bound reference oligonucleotide probe based on said known reference sequence and a set of surface bound deletion oligonucleotide probes of at least about 20 nucleotides in length wherein said set of deletion oligonucleotide probes is prepared by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe,
(b) determining hybridization ratios for said set of deletion oligonucleotide probes with respect to said reference oligonucleotide probe and
(c) relating said hybridization ratios to the presence or absence of differences between said individual sequence and said reference sequence.

37. An addressable array comprising:
(a) a support having a surface,
(b) a spot on said surface having bound thereto an oligonucleotide probe specific for a target nucleic acid sequence and
(c) at least one spot on said surface having bound thereto a cross-hybridization oligonucleotide probe wherein said cross-hybridization oligonucleotide probe measures the extent of the occurrence of a cross-hybridization event of a predetermined probability between an interfering nucleic acid sequence and said oligonucleotide probe specific for a target nucleic acid sequence wherein said cross-hybridization oligonucleotide probe is selected by a process comprising deletion of at least one nucleotide from a single hybridization oligonucleotide wherein said deletion(s) are evenly spaced with respect to the nucleotides of said hybridization oligonucleotide and the number of deletions is based on the length of said cross-hybridization oligonucleotide probe.

38. A method for detecting a target nucleic acid sequence, said method comprising:
(a) contacting a medium suspected of containing said target nucleic acid sequence with the array of claim 37 and
(b) determining a result of said contacting, said result indicating the presence or absence of said target nucleic acid sequence in said medium.

39. A method according to claim 38 wherein said determining of said result comprises examining said array for the presence of a hybrid of said target nucleic acid sequence and said oligonucleotide probe specific for said target nucleic acid sequence, the presence thereof indicating the presence of said target nucleic acid sequence in said medium.

40. An assay result determined by a method according to claim 38.

41. A method comprising forwarding to a remote location a result according to claim 40.

42. An addressable array comprising:
(a) a support having a surface,
(b) a spot on said surface having bound thereto an oligonucleotide probe specific for a target nucleic acid sequence and
(c) at least one spot on said surface having bound thereto a cross-hybridization oligonucleotide probe wherein said cross-hybridization oligonucleotide probe measures the extent of the occurrence of a cross-hybridization event of a predetermined probability between an interfering nucleic acid sequence and said oligonucleotide probe specific for a target nucleic acid sequence wherein said cross-hybridization oligonucleotide probe is selected by a method according to claim 26.

43. A method for detecting a target nucleic acid sequence, said method comprising:
(a) contacting a medium suspected of containing said target nucleic acid sequence with the array of claim 42 and
(b) determining a result of said contacting, said result indicating the presence or absence of said target nucleic acid sequence in said medium.

44. An assay result determined by a method according to claim 43.

45. A method comprising forwarding to a remote location a result according to claim 44.

46. A method for predicting the potential of a hybridization oligonucleotide of at least about 20 nucleotides in length to hybridize to a complementary target nucleotide sequence, said method comprising:
(a) obtaining, from a nucleotide sequence at least about 30 nucleotides in length L and complementary to said target nucleotide sequence, a set of overlapping oligonucleotides of at least about 20 nucleotides in length and of identical length N and spaced S nucleotides apart, said set comprising 1+Int[(L−N)/S] oligonucleotides, wherein “Int” is the integer part of the indicated quotient,
(b) determining experimentally and evaluating for each of said oligonucleotides the hybridization of said oligonucleotides with said target nucleotide sequence,
(c) selecting a subset of oligonucleotides within said predetermined number of oligonucleotides based on said evaluation and application of a rule that rejects some of said oligonucleotides of step (b),
(d) identifying oligonucleotides in said selected subset, viewed according to order of position along said nucleotide sequence, that are in clusters along a region of said nucleotide sequence,
(e) ranking said clusters in order of number of oligonucleotides in each cluster from greatest number to least number of oligonucleotides,
(f) selecting for each cluster, in descending order of cluster rank, a hybridization oligonucleotide of predetermined length that has as its central nucleotide the central nucleotide of a region of said nucleotide sequence that corresponds to said cluster, the remaining nucleotides of said hybridization oligonucleotide being added, in correspondence to the nucleotides in said nucleotide sequence that extend in one or both directions from said central nucleotide, until said hybridization oligonucleotide is obtained and a predetermined number of hybridization oligonucleotides are obtained, one from each cluster, wherein the higher the rank of said cluster, the higher the hybridization potential of said hybridization oligonucleotide for said target nucleotide sequence and wherein said predetermined length of said hybridization oligonucleotide is greater than the length of any of said oligonucleotides in said cluster.
